(19) United States
(12) Patent Application Publication
(10) Pub. No.: US 2012/0238487 A1
(43) Pub. Date: Sep. 20, 2012

(54) POLYMER COMPOSITION USEFUL AS A PHARMACEUTICAL CARRIER

(75) Inventors: Paul Kong Thoo Lin, Aberdeen (GB); Woei Ping Cheng, Welwyn (GB); Clare Hoskins, St Albans (GB)

(73) Assignee: ROBERT GORDON UNIVERSITY, SCHOOL HILL, ABERDEEN, ABERDEENSHIRE (GB)

(21) Appl. No.: 13/394,117

(22) PCT Filed: Sep. 6, 2010
(86) PCT No.: PCT/GB2010/051477

§ 371 Date: Jun. 11, 2012

(30) Foreign Application Priority Data
Sep. 4, 2009 (GB) 0915449.3

(51) Int. Cl. A61K 47/34; C08F 228/02; A61K 31/7088; C08F 222/38; C08B 37/00; A61K 38/02

(52) U.S. Cl. 514/1.1; 536/55.1; 514/44 R; 514/772.1; 525/328.4

(57) ABSTRACT

A polymer having a structure according to the following formula (I): wherein: A represents a hydrophilic group; B represents a hydrophobic aromatic group such as 5-dimethylamino-1-naphthalenesulfonyl (Dansyl), 9-fluorenylmethoxy carbonyl (Fmoc) and naphthalene (Naphth); D and E independently represent amine groups; F represents an amine group, the amine group being substituted with a B group and an A group, or the amine group being a quaternary ammonium moiety; wherein W, X, Y and Z each independently have values greater than or equal to (1), especially in the range of (1) to (10).

The present invention relates to a new polymer and the use of the polymer as a delivery system for substantially hydrophobic entities such as substantially hydrophobic drugs, proteins or peptides. Alternatively the polymer may be used in the delivery of DNA. Typically the delivery system of the present invention increases the water solubility of hydrophobic entities and enhances the cell uptake of such entities.

Many modern drugs are substantially water-insoluble. Approximately 40% of all new drugs in the developmental stage are hydrophobic. This unfavourable physico-chemical property presents a challenge for drug formulators and often results in the failure of such drugs at the development stage. Substantially water insoluble or hydrophobic drugs are also difficult to administer and their bioavailability is low.

New formulation technologies are required to effectively administer hydrophobic drugs. Traditionally low molecular weight surfactants, liposomes and co-solvents were used however all these technologies require a high ratio of excipient to drug making them inefficient.

Formulation systems containing a surfactant such as Cremophor EL are known. Such formulation systems often however cause an adverse reaction in the patient which may prevent or limit the success of the drug. Furthermore, the ratio of drug to excipient in these formulations is typically low and only limited amounts of the drug will be solubilised within the drug delivery system. Accordingly, relatively large amounts of the drug formulation must be administered to ensure administration of the required amount of the drug. The large dosage regimes limit patient compliance, limiting the therapeutic effectiveness of such drug formulations. Unpleasant and potentially dangerous side effects are often caused through the administration of hydrophobic drugs with a narrow therapeutic index when using conventional formulations. Such side effects are often caused by the lack of tissue specificity of conventional formulations.

Known formulations for the delivery of substantially hydrophobic or water insoluble entities are commonly unstable and have a limited shelf life. Additionally the price and availability of known delivery systems, or components thereof, make the use of such delivery systems unfavourable.

As detailed above, the challenges associated with the administration of substantially hydrophobic entities such as hydrophobic drugs are well known. Attempts to overcome these challenges through the use of amphiphilic polymers comprising a polyethyleneimine (PEI) backbone having pendant hydrophilic and hydrophobic groups attached thereto has been reported in WO 2004/026941. The same publication also reports that the PEI polymer may be used to administer a poorly water soluble drug.

The use of amphiphilic polymers comprising a polyallylamine (PAA) backbone having pendant hydrophilic and hydrophobic groups, the hydrophobic group being a hydrocarbon chain, particularly cholesterol, palmitoyl or cetyl, has been reported in WO2008/007092.

Efficient drug delivery is only one of a number of requirements of a successful medical treatment. It is often beneficial and sometimes necessary to know where the administered drug is in the patient's body. This is especially useful for anti-cancer drugs that must be targeted at a particular area of tissue of particular cells. To achieve this it is necessary for the drug or its delivery system to include means by which it can be detected or its position monitored from outside the patient's body. According to a first aspect of the present invention there is provided a polymer having a structure according to the following formula:

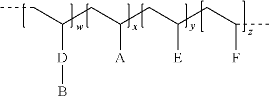

wherein:
A represents a hydrophilic group;
B represents a hydrophobic group;
D and E independently represent amine groups;
F represents an amine group, the amine group being substituted with a B group and an A group, or alternatively the amine group being a quaternary ammonium moiety; wherein W, X, Y and Z have values greater than or equal to 1;
characterised in that the hydrophobic group B is an aromatic group.

Optionally the values of W, X, Y and Z are each independently between 1 and 50; typically between 1 and 25 and preferably between 1 and 10.

Optionally the molar ratio of monomeric unit Z to monomeric unit Y is 0:100 and the molar ratio of monomeric unit W to monomeric unit Y is 0.01 to 100:100.

Optionally the molar ratio of monomeric unit X to monomeric unit Y is 0 to 100:100.

Typically D and E are independently primary alkylamine groups.

Optionally D is disubstituted.

Optionally D is substituted with two B groups.

Optionally one or more of the molar ratio of monomeric units Y and Z is zero.

In a particular embodiment the polymer comprises only monomeric units W and X.

Typically the polymer is based upon a polyallylamine (PAA) polymer. Advantageously the polymer is cationic.

Optionally the cationic polymer is relatively non-toxic and therefore suitable for use in delivery of a drug in an animal or human body.

Generally the polymer has a molecular weight of between 10 and 50 kDa, typically 15 kDa.

Advantageously the polymer is amphiphilic.

Typically the amphiphilic polymer comprises one more of hydrophilic, lipophilic and hydrophobic moieties.

Typically the polymer forms nano self-assemblies in aqueous media.

Typically the polymer forms a hydrophobic core upon contact with an aqueous media due to the aggregation of the hydrophobic moieties.

Advantageously the hydrophobic core provides a ‘micro-container’ or ‘envelope’ for molecules, in particular hydrophobic molecules.

Typically these self-assemblies suitably consist of polymeric micelles, polymeric nanoparticles or polymeric vesicles.

Preferably the polymer contains a chromophore.

Advantageously the aromatic group of hydrophobic group B comprises the chromophore.

Typically the position of the chromophore can be externally monitored following administration of the polymer to a human or animal.

Generally the polymer can be externally detected and its position in a human or animal body monitored externally.

Preferably the polymer is fluorescent.

Typically the aromatic group of hydrophobic group B is fluorescent.

Advantageously the fluorescence of the polymer is used for one or more of the study of cell biology, in vivo imaging and biological diagnosis.

The arrangement of the monomeric units W, X, Y and Z may be in any order, and the relative positions of the hydrophilic and hydrophobic attachments are therefore random.

Preferably no more than three consecutive monomeric units are the same.

In one embodiment the molar ratio of monomeric unit W to monomeric unit Y is 0.01 to 60:100; suitably 1 to 20:100; more suitably 1 to 10:100; advantageously 1 to 5:100.

In one embodiment the molar ratio of monomeric unit X to monomeric unit Y is 0.01 to 100:100; typically 10 to 90:100; suitably 30 to 70:100; more suitably 40 to 60:100; advantageously 40 to 90:100.

In one embodiment the molar ratio of monomeric unit Z to monomeric unit Y is 0.01 to 60:100; suitably 1 to 20:100; more suitably 1 to 10:100; advantageously 1 to 5:100.

In one embodiment the polymer has a structure according to the following formula:

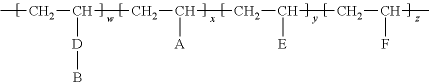

wherein:
D represents CH₂—NH;
B represents a hydrophobic, aromatic group;
A represents CH₂-hydrophilic group;
E represents CH₂—NH₂;
F represents

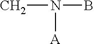

or a quaternary ammonium moiety wherein one of the substituents of the quaternary ammonium moiety is a B group.

Typically the PAA polymer upon which the polymer is based has an average molecular weight of between 10 to 70 kD; suitably 10 to 25 kD; advantageously 15 kD.

Preferably the hydrophobic, aromatic group B is one or more of a bi-cyclic ring system, a tri-cyclic ring system, a phenyl group and alkylbenzene group.

Optionally one or more of the bi-cyclic ring system, tri-cyclic ring system, phenyl group and alkylbenzene group is substituted.

Typically the substituent is one or more of an alkenyl, alkynyl, acyl, hydroxy alkyl, hydroxy acyl or sugar group.

Optionally the aromatic group is flexible.

Typically the hydrophilic group is an amine.

Optionally the CH₂ group linking the hydrophilic group to the polymer backbone is an alkyl group.

Typically the amine is a primary, secondary or tertiary amine.

Optionally the primary, secondary or tertiary amine is substituted with an additional hydrophilic group.

Typically the additional hydrophilic group is a non-ionic group such as methyl glycolate or polyethylene glycol.

Alternatively the additional hydrophilic group is one or more of a hydrogen, alkyl, alkenyl, alkynyl, aryl, acyl, hydroxy alkyl, hydroxy acyl, polyethylene glycol or sugar group.

Where appropriate the substituents listed above may be in linear, branched, substituted, unsubstituted or cyclic form.

Preferably the amine groups listed above are substituted with one or more sugar groups comprising 1 to 20 carbon atoms; more suitably 1 to 12 carbon atoms; typically 1 to 6 carbon atoms.

Optionally the hydrophilic group represents a quaternary ammonium moiety typically having the structure:

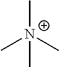

Preferably the quaternary ammonium moiety is attached to the carbon backbone of the PAA polymer via an alkyl group such as CH₂.

Typically the other three groups attached to the quaternary ammonium moiety are independently one or more of a hydrogen, alkyl, alkenyl, alkynyl, aryl, acyl, hydroxy alkyl, hydroxy acyl, polyethylene glycol and sugar group comprising between 1 and 6 carbon atoms.

Optionally the hydrophobic aromatic group B is 5-Dimethylamino-1-naphthalenesulfonyl (Dansyl), where Dansyl typically has the structure:

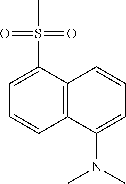

Advantageously the hydrophobic aromatic group B is 9-Fluorenylmethoxy carbonyl (Fmoc), where Fmoc has the structure:

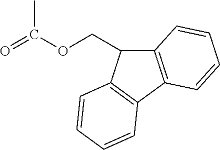

Advantageously the hydrophobic aromatic group B is Naphthalene (Naphth), where Naphth has the structure:

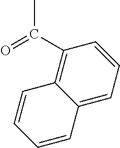

As noted above, D and E independently comprise an amine group, typically a primary alkyl amine group.

Preferably the amine group has the structure CH₂NHR or CH₂NH₂ where R represents a substituted or unsubstituted hydrocarbon chain. R may represent hydrophobic group B.

Alternatively D and E independently represent one or more of a sulphonamide, amide, hydrazine, oxime and oxycarbonyl linker.

Optionally the carbon backbone of the polymer is substituted or unsubstituted.

In one embodiment the carbon backbone of the polymer is unsubstituted. Suitably the carbon backbone of the polymer, in combination with groups D and E, consists solely of primary amines.

Optionally the carbon backbone of the polymer has the structure:

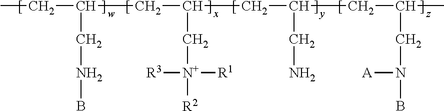

wherein A and B are as described above.

Typically R¹, R² and R³ independently represent a hydrogen, alkyl, alkenyl, alkynyl, aryl, acyl, hydroxy alkyl, hydroxy acyl, polyethylene glycol or sugar group.

Preferably the polymer is in the form of a solution, typically an aqueous solution. Alternatively the polymer is in the form of a freeze-dried composition.

Where the polymers of the present invention are amphiphilic they consist of hydrophobic and hydrophilic moieties within the same macromolecule and the polymers generally form nano self-assemblies in aqueous media. A hydrophobic core is suitably created upon contact with an aqueous media due to the aggregation of the hydrophobic moieties. The hydrophobic core can serve as a “micro-container” for molecules, in particular hydrophobic molecules. These self-assemblies suitably consist of polymeric micelles, polymeric nanoparticles or polymeric vesicles.

Advantageously the amphiphilic polymer self-assembles in aqueous solution above a critical aggregation concentration. This concentration is lower than that of traditional surfactant micelles. The aggregation of the amphiphilic polymer is stable in aqueous solution, allowing it to circulate in the bloodstream of a patient without dissociating.

According to a further aspect of the present invention the polymer has the structure:

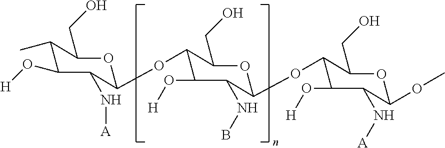

wherein A represents a CH₂-hydrophillic group; and B represents a hydrophobic, aromatic group.

According to a further aspect of the present invention there is provided a composition comprising a polymer of the present invention and a pharmaceutically acceptable vehicle. Suitable pharmaceutically acceptable vehicles are well known to those skilled in the art and include aqueous and non-aqueous solutions, emulsions and suspensions.

Generally the non-aqueous solutions, emulsions and suspensions include non-aqueous solvents that may be water-miscible such as propylene or polyethylene glycol, oils such as vegetable oils, or organic esters. Aqueous pharmaceutically acceptable vehicles include alcoholic/aqueous solutions, emulsions or suspensions including saline, particularly 0.9% weight/volume (w/v) saline.

Typically the aqueous pharmaceutically acceptable vehicle comprises distilled water.

Preferably the composition comprises an aqueous pharmaceutically acceptable vehicle.

Optionally the composition may also comprise additives such as preservatives, antimicrobials, antioxidants and chelating agents.

Typically the ratio of polymer of the present invention to pharmaceutically acceptable vehicle by weight/volume (w/v) (g/ml) is 0.0001-100:100; typically 0.001 to 10:100; advantageously 0.01-1:100.

Typically when contacted with an aqueous media, the hydrophobic groups of the polymer aggregate to form hydrophobic solubilising domains within the aqueous media.

Optionally the composition may be formed by mixing the polymer described above and a pharmaceutically acceptable vehicle, suitably an aqueous pharmaceutically acceptable vehicle.

Typically the composition is formed using probe sonication.

Typically the composition is stable for two or more months at room temperature.

Preferably the composition is a substantially homogenous composition and remains homogenous upon storage for two or more months.

According to a further aspect of the present invention there is provided a delivery composition comprising the composition described above and an entity to be delivered, said entity typically having a limited solubility in an aqueous media.

Typically the entity is substantially or completely water insoluble, in general the entity is substantially or completely hydrophobic.

Alternatively the entity may have a limited solubility in a non-aqueous media such as oil.

Typically the entity is substantially or completely lipophilic.

Typically the entity has an aqueous solubility of 0.001 to 0.2 mg/ml at a temperature of 15 to 25° C.

Typically the entity is a drug, peptide, protein or polymer.

Where the entity is a drug, the drug is suitably a steroid such as prednisolone, oestradiol or testosterone; a drug having a multicyclic ring structure lacking polar groups such as paclitaxel, griseofulvin, amphotericin B, propofol, or etoposide; or anticancer drugs such as bis-naphthalimidopropylalkylamines.

Where the entity is a peptide, the peptide is suitably a therapeutic enzyme or hormone such as glucagon or cyclosporine.

Where the entity is a protein, the protein is suitably a therapeutic enzyme or hormone such as insulin.

Typically the entity is DNA which has a relatively high solubility in an aqueous media, but a limited non-aqueous solubility.

Where the entity is DNA, the delivery composition preferably exhibits excellent DNA binding and condensing properties.

Typically in an aqueous media, the entity is suitably housed or encapsulated within the hydrophobic solubilising domains formed from the aggregation of the hydrophobic groups of the polymer.

Preferably the delivery composition allows delivery of the entity into the body of an animal or human.

Typically the delivery composition is deliverable orally or parenterally including via a subcutaneous, intramuscular, intravenous or intrathecal route. Alternatively, the delivery composition is deliverable via a rectal, vaginal, ocular, sublingual, nasal, pulmonary or transdermal route.

Typically the ratio of entity to polymer by weight is typically 0.001 to 100:100; suitably 1 to 100:100; more suitably 10 to 90:100; generally 30 to 70:100.

Preferably the delivery composition is in the form of a solution, tablet, suppository, capsule, powder, emulsion, gel, foam or spray.

Typically the solution is transparent, translucent or opaque.

Typically the delivery solution is coloured.

According to a further aspect of the present invention there is provided the delivery composition as described above for use in therapy.

According to a further aspect of the present invention there is provided the use of the delivery composition as described above in the manufacture of an anaesthetic or a medicament for the treatment of an infection or a disease such as cancer, diabetes, cardiovascular disease, hereditary diseases, metabolic diseases, or bacterial and viral infections.

According to a further aspect of the present invention there is provided a method of treatment comprising the administration of the delivery composition described above to an animal or human patient in need of treatment.

According to a further aspect of the present invention there is provided a method of increasing the solubility of an entity having limited solubility in a media comprising the steps of mixing the entity, a polymer as described above and the media together to form a solution.

The media typically comprises a pharmaceutically acceptable carrier such as those listed above. Typically the media is an aqueous media. The aqueous media may be in the form of one or more of an aqueous solution, suspension or emulsion or an alcohol/aqueous solution, suspension or emulsion including saline and buffered media.

The entity having limited solubility in aqueous media is suitably a drug, polymer, peptide or protein.

Typically the entity has limited solubility in an aqueous media; typically the entity has an aqueous solubility of 0.001 mg/ml to 0.2 mg/ml at a temperature of 15 to 25° C.

Alternatively the entity may have a limited solubility in a non-aqueous media. Typically the entity is DNA.

Typically the polymer is mixed with the media prior to mixing with the entity.

Alternatively the polymer is mixed with the entity prior to mixing with the media.

Typically the polymer is mixed with the entity at a ratio of 0.001 to 100:100 by weight; generally 30 to 70:100 by weight.

Advantageously the drug loading ratio of polymer to entity is 1:2, preferably 1:10 and optionally lower.

Typically the drug solubility increases with higher drug loading concentrations. Accordingly, the drug solubility at low polymer to entity ratios is greater than the drug solubility at high polymer to entity ratios. Higher drug loading concentrations are also associated with the achievement of optimum therapeutic effects. Typically the polymer is added to the media, suitably aqueous media, at a ratio of 0.001 to 100:1 w/v; generally at a concentration of 0.01 to 1:1 w/v. Advantageously, the solubility of the entity is increased at least five fold; typically at least ten fold; suitably at least twenty fold; more suitably twenty-four fold or more.

According to a further aspect of the present invention there is provided a method of promoting the absorption of an entity having limited solubility in an aqueous media comprising the steps of mixing the entity, a polymer as described above and an aqueous media.

Preferably the absorption of the entity by the human or animal body is maximised.

Typically the ability of the entity to cross biological barriers, in particular cell barriers is maximised and the uptake of the entity in vivo or in culture cells is facilitated.

Typically the toxicity of the entity to an animal or human body is reduced when the entity is mixed with a polymer as described above.

Preferably the efficiency of the entity or a therapeutic agent is enhanced.

Typically the bioavailability and/or reduced toxicity of the entity or therapeutic agent enhance the efficiency.

Preferably the release of the entity is prolonged over a period of up to 72 hrs; typically the period is up to 96 hrs.

The entity may suitably be a drug, protein, polymer, peptide or DNA.

As noted above, upon contact with an aqueous media the hydrophobic groups of the polymer described above aggregate to form hydrophobic solubilising domains or cores. The hydrophobic solubilising domains typically protect the entity from degradation following administration.

According to one embodiment of the present invention the entity is DNA. A complex is typically formed upon contact of the polymer described above with DNA through the electrostatic attraction between the amine groups (D and E) of the polymer and the phosphate groups of the DNA molecules. The complex is very stable and suitably protects the DNA molecules from degradation following administration, in particular through administration via injection. Typically the ability of the DNA to cross biological barriers is maximised to allow the DNA entry into the nucleus of a cell and facilitate the uptake of the DNA.

According to a further aspect of the present invention there is provided a method of producing the drug delivery composition described above comprising the steps of mixing the polymer described above, an entity to be delivered having limited solubility in a media and the media.

Typically the mixing step involves the use of probe sonication.

Optionally the polymer is mixed with an aqueous media prior to mixing with the entity.

Alternatively the polymer may be mixed with the entity prior to mixing with the aqueous media.

The structure of the drug delivery composition so formed may be analysed and verified using any suitable technique. For instance, the PAA polymer in the delivery composition may be analysed and characterised using IR, ¹H and ¹³C NMR or elemental analysis.

According to a further aspect of the present invention there is provided a nanosystem comprising the composition described above and a hydrophobic substance, the nanosystem being an environmentally benign solvent.

Optionally the nanosystem is a stable micellar system.

Typically the micellar system enhances the stability of an emulsion.

Typically the emulsion is used in the manufacture of one or more of paper, paint and foodstuffs.

The invention will now be described by way of example only having reference to the accompanying Figures in which:

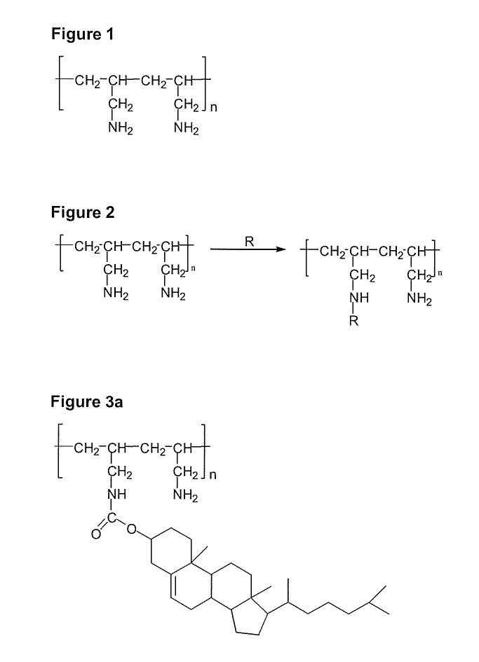

FIG. 1 shows the chemical structure of polyallylamine (PAA);

FIG. 2 shows the general reaction schematic for PAA with hydrophobic and hydrophilic grafting where R=Cholesterol, 5-Dimethylamino-1-naphthalenesulfonyl, 9-Fluorenylmethoxy carbonyl or Naphthalene;

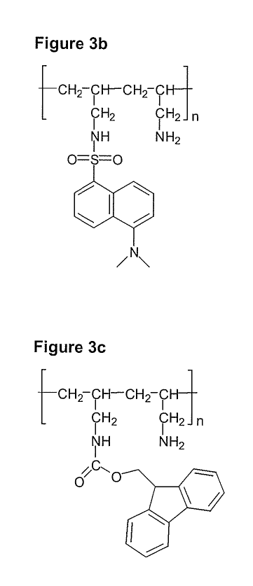

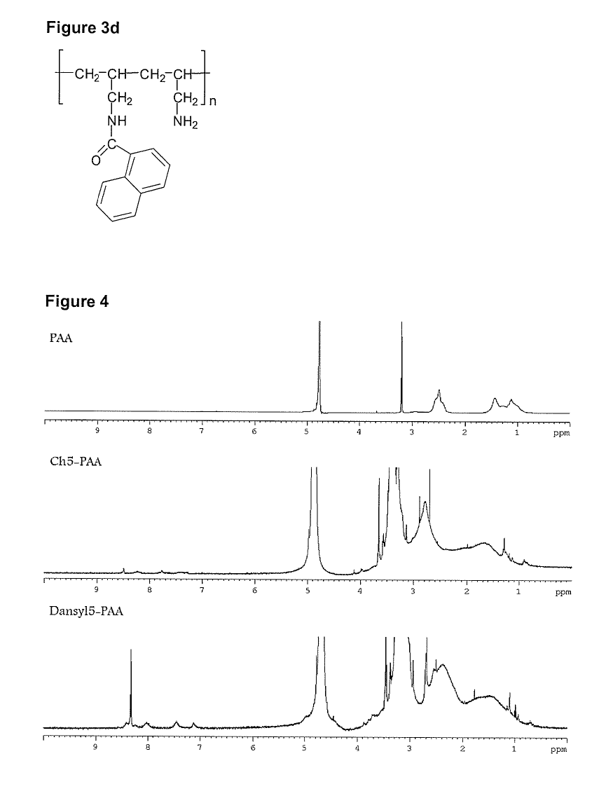

FIGS. 3a, 3b, 3c and 3d show the chemical structure of (3a) Cholesterol-PAA (Ch-PAA); (3b) 5-Dimethylamino-1-naphthalenesulfonyl-PAA (Dansyl-PAA); (3c) 9-Fluorenylmethoxy carbonyl-PAA (Fmoc-PAA); and (3d) Naphthalene-PAA (Naphth-PAA) respectively;

FIG. 4 shows an ¹H NMR spectra of PAA, Ch-PAA and Dansyl-PAA;

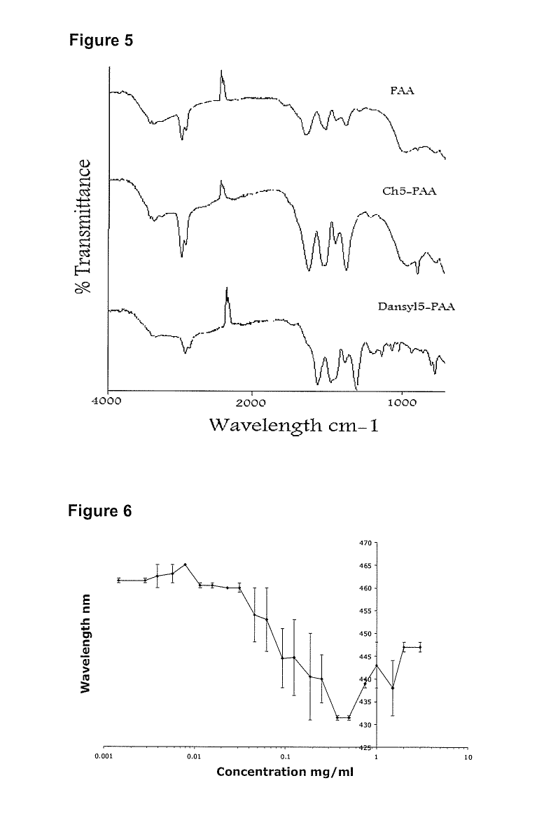

FIG. 5 shows an FTIR spectra of PAA, Ch-PAA and Dansyl-PAA;

FIG. 6 shows the effect of the concentration of Ch-PAA on the peak absorbance of methyl orange at 464 nm;

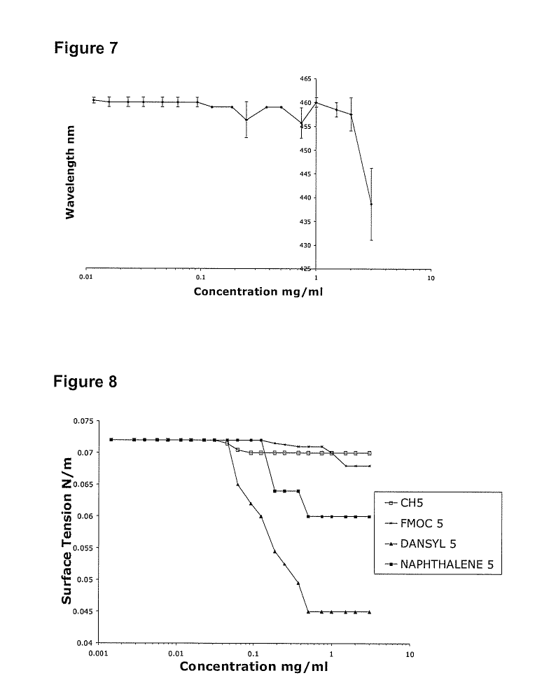

FIG. 7 shows the effect of the concentration of Fmoc-PAA on the peak absorbance of methyl orange at 464 nm;

FIG. 8 shows surface tension results for 5% mole substituted modified polymers;

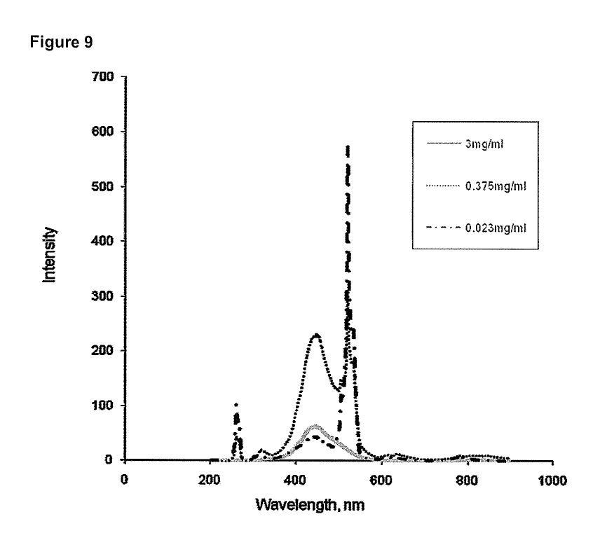

FIG. 9 shows fluorescent spectra of Fmoc-PAA scanned between 200-900 nm;

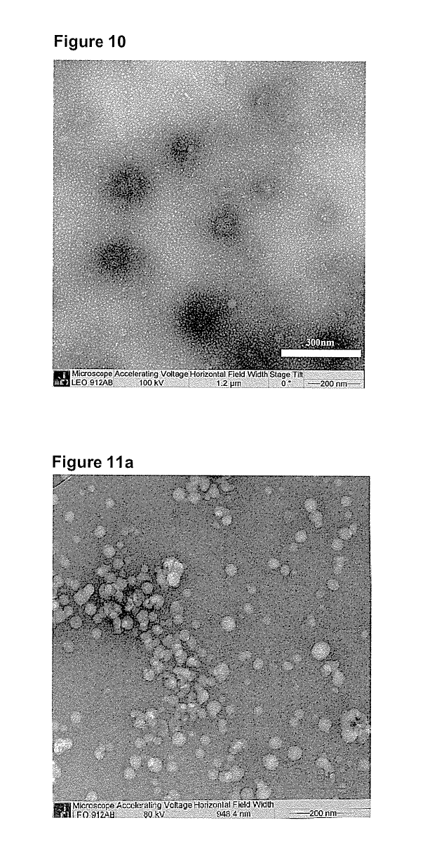

FIG. 10 shows a TEM analysis of Ch-PAA;

FIGS. 11a & 11b show a TEM analysis of Fmoc-PAA at (a) 0.4 mgmL⁻¹ and (b) 2 mgmL⁻¹ respectively;

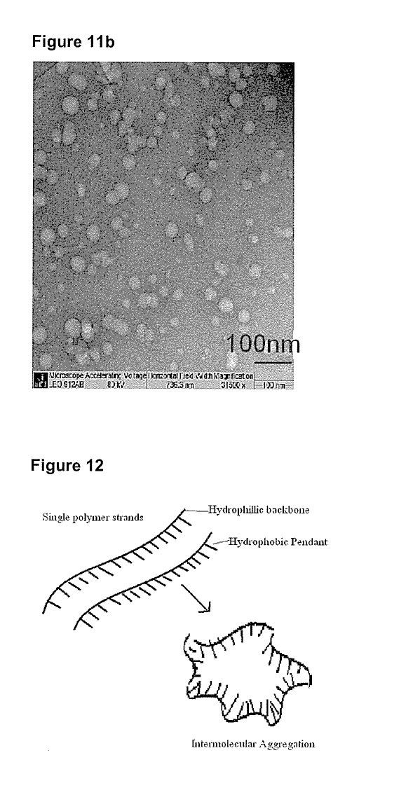

FIG. 12 shows a polymer aggregation model for Ch-PAA and Dansyl-PAA;

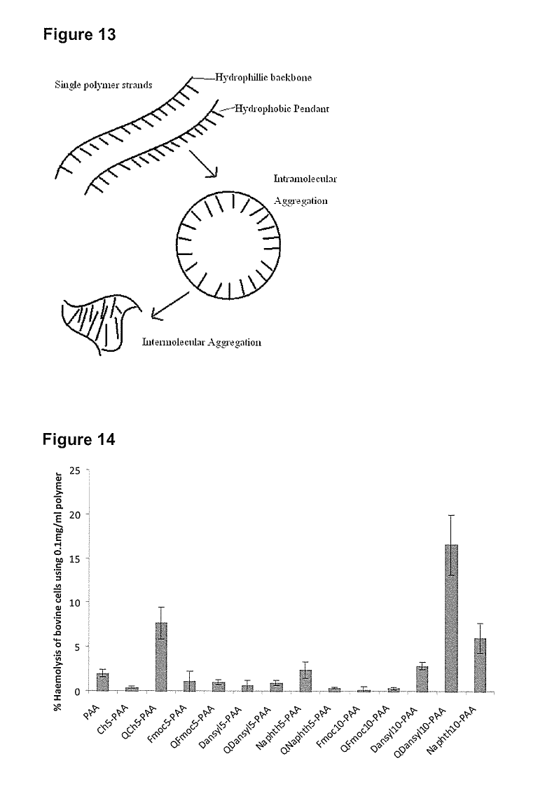

FIG. 13 shows a polymer aggregation model for Fmoc-PAA and Naphth-PAA;

FIG. 14 shows the percentage haemolysis of bovine cells using 0.1 mg/ml polymer;

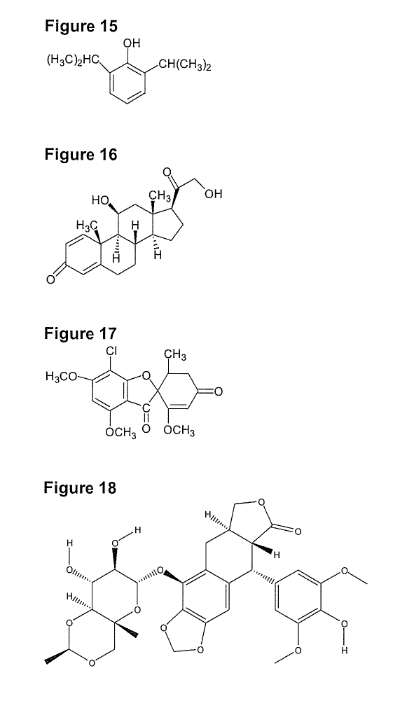

FIG. 15 shows a chemical structure of Propofol;

FIG. 16 shows a chemical structure of Prednisolone;

FIG. 17 shows a chemical structure of Griseofulvin;

FIG. 18 shows a chemical structure of Etoposide;

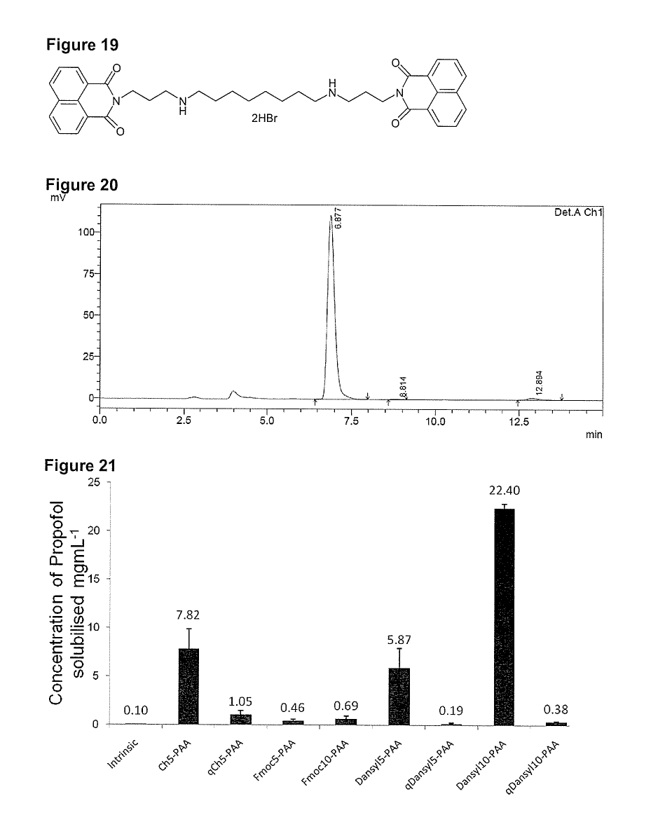

FIG. 19 shows a chemical structure of BNIPDaoct;

FIG. 20 shows a reverse phase HPLC chromatogram of propofol detected at 229 nm;

FIG. 21 shows the maximum concentration of propofol solubilised by each modified polymer determined by HPLC, compared to the intrinsic solubility, n=3;

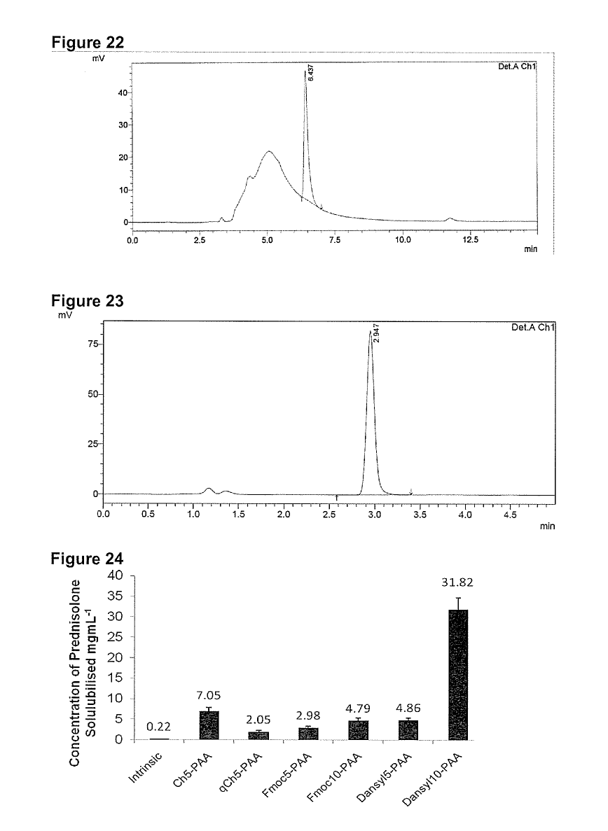

FIG. 22 shows an HPLC spectra of Naphth₅ and propofol formulation showing peak overlap at propofol retention time;

FIG. 23 shows a reverse phase HPLC chromatogram of prednisolone detected at 243 nm;

FIG. 24 shows the maximum concentration of prednisolone solubilised by each modified polymer determined by HPLC, compared to the intrinsic solubility, n=3;

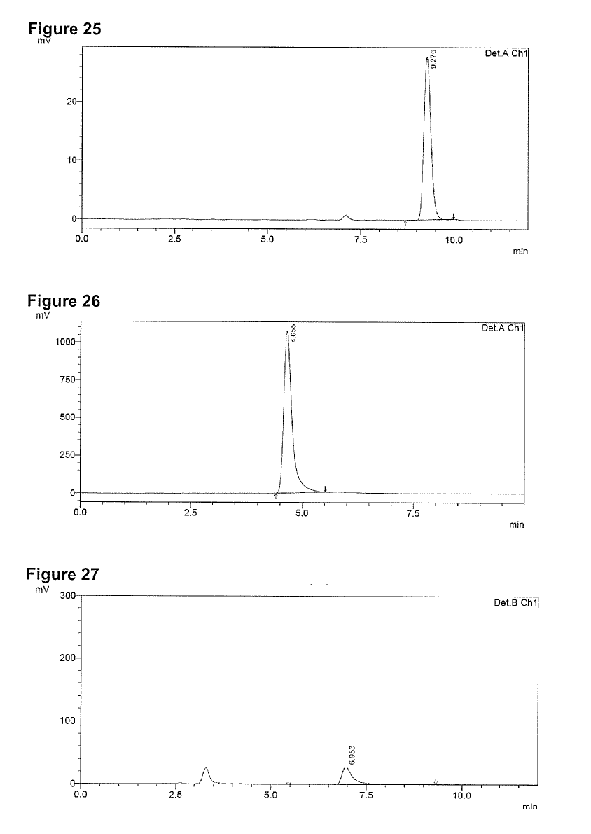

FIG. 25 shows a reverse phase HPLC chromatogram of Griseofulvin detected at 293 nm;

FIG. 26 shows a reverse phase HPLC chromatogram of etoposide detected at 229 nm;

FIG. 27 shows a reverse phase HPLC chromatogram of BNIPDaoct detected at 394 nm;

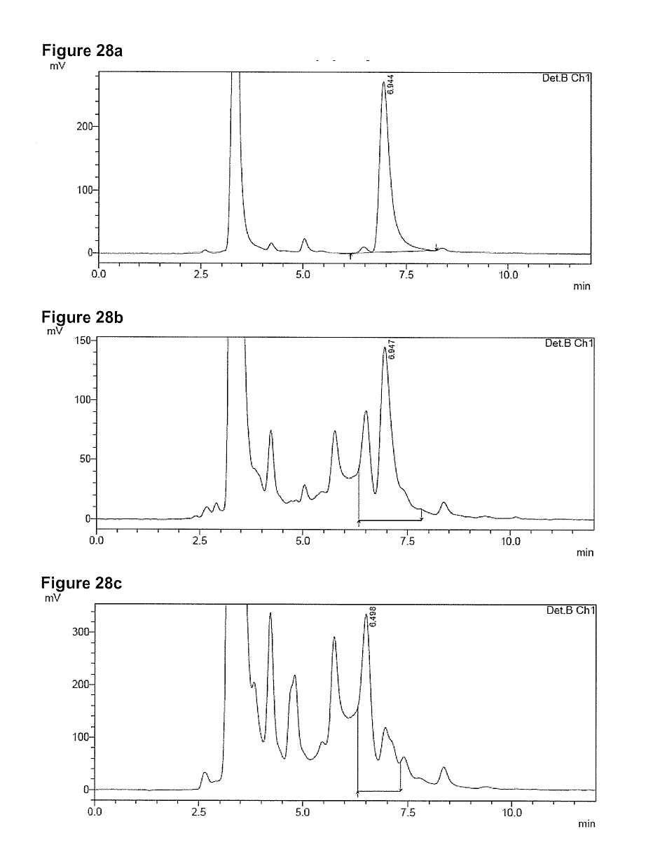

FIG. 28 shows an HPLC spectra for 1 mgmL⁻¹ Ch₅ a) 1:1 b) 5:1 c) 10:1 drug:polymer ratio with BNIPDaoct;

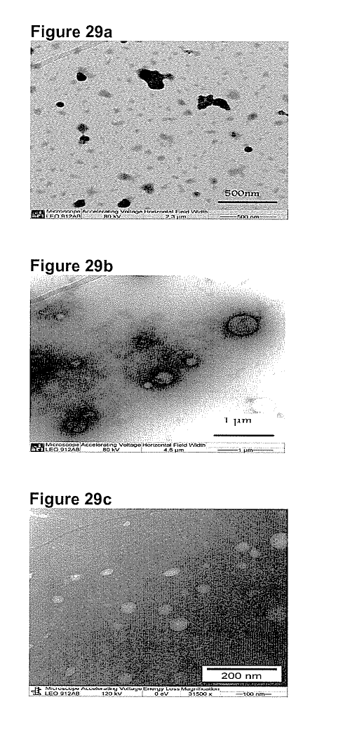

FIG. 29 shows a TEM imaging of a) 6 mgmL⁻¹ Ch₅, b) 6 mgmL⁻¹ Ch₅+10:1 propofol, c) 6 mgmL⁻¹ Ch₅+10:1 prednisolone, and d) 6 mgmL⁻¹ Ch₅+10:1 griseofulvin;

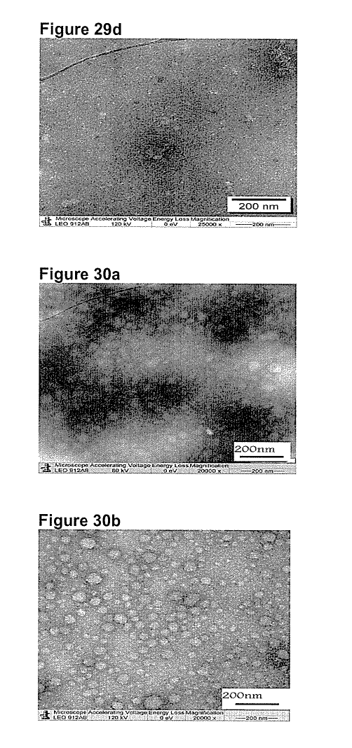

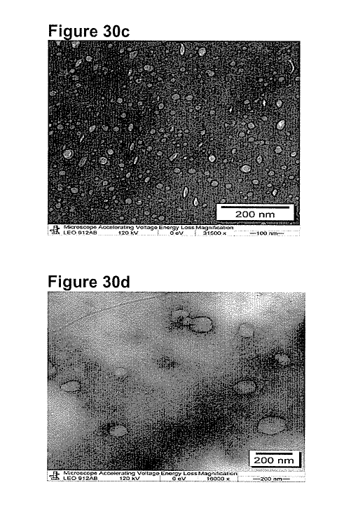

FIG. 30 shows a TEM imaging of a) 6 mgmL⁻¹ Dansyl₁₀, b) 6 mgmL⁻¹ Dansyl₁₀+10:1 propofol, c) 6 mgmL⁻¹ Dansyl₁₀+10:1 prednisolone, and d) 6 mgmL⁻¹ Dansyl₁₀+10:1 griseofulvin;

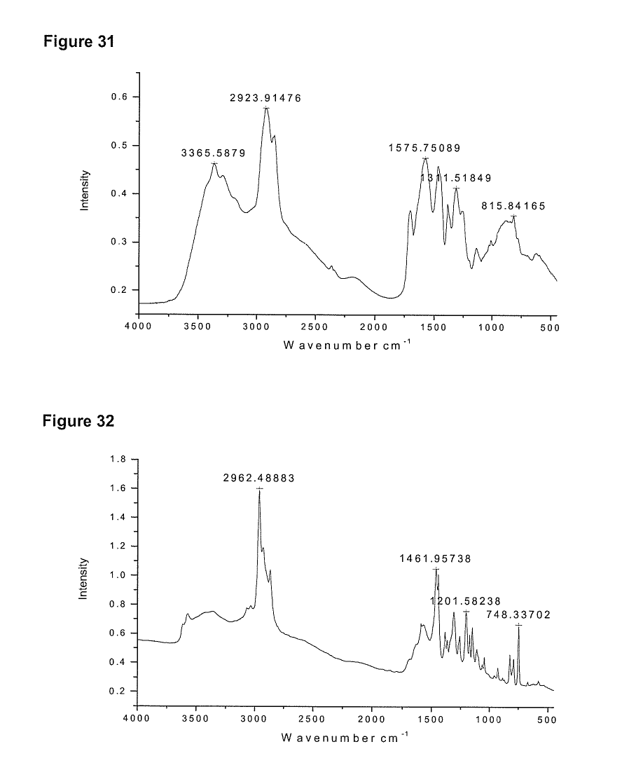

FIG. 31 shows an FTIR spectra of Ch₅;

FIG. 32 shows an FTIR spectra of Ch₅+propofol;

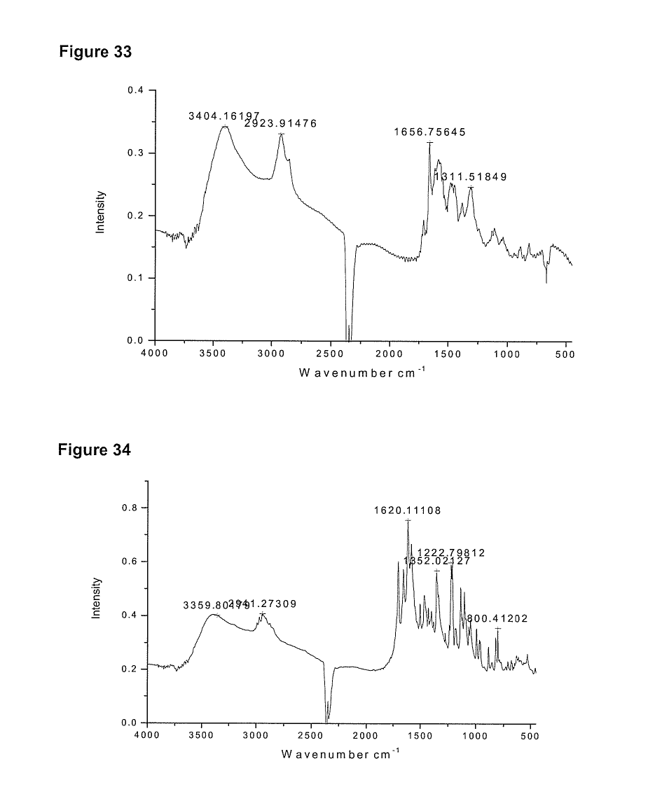

FIG. 33 shows an FTIR spectra of Ch₅+prednisolone;

FIG. 34 shows an FTIR spectra of Ch₅+griseofulvin;

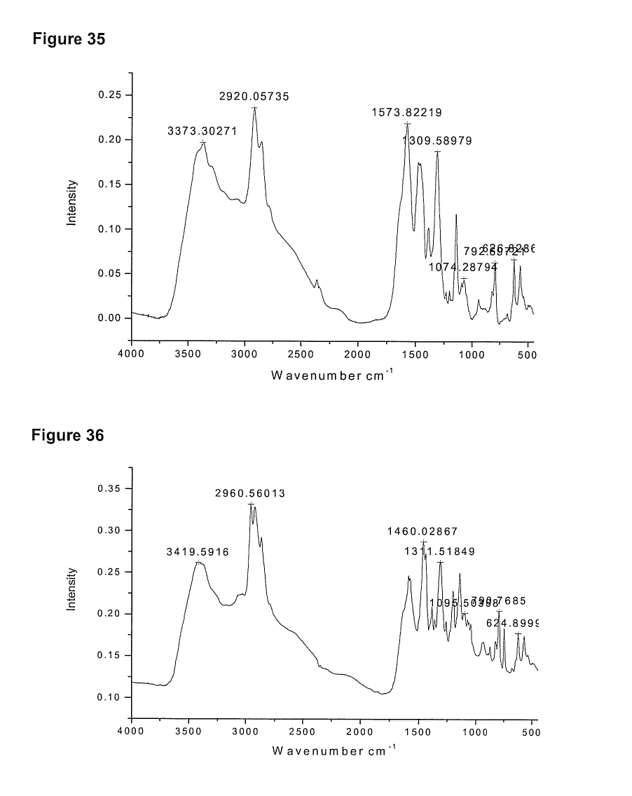

FIG. 35 shows an FTIR spectra of Dansyl₁₀;

FIG. 36 shows an FTIR spectra of Dansyl₁₀+propofol;

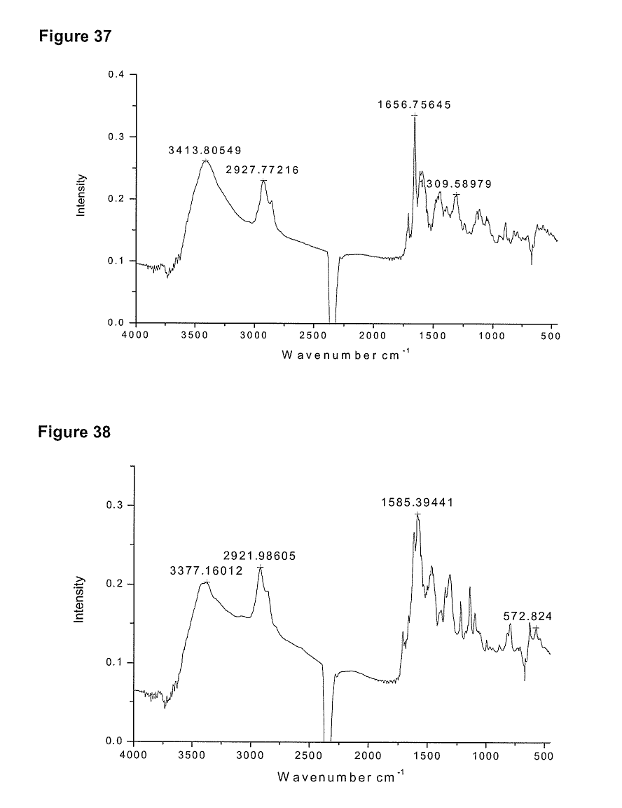

FIG. 37 shows an FTIR spectra of Dansyl₁₀+prednisolone;

FIG. 38 shows an FTIR spectra of Dansyl₁₀+griseofulvin;

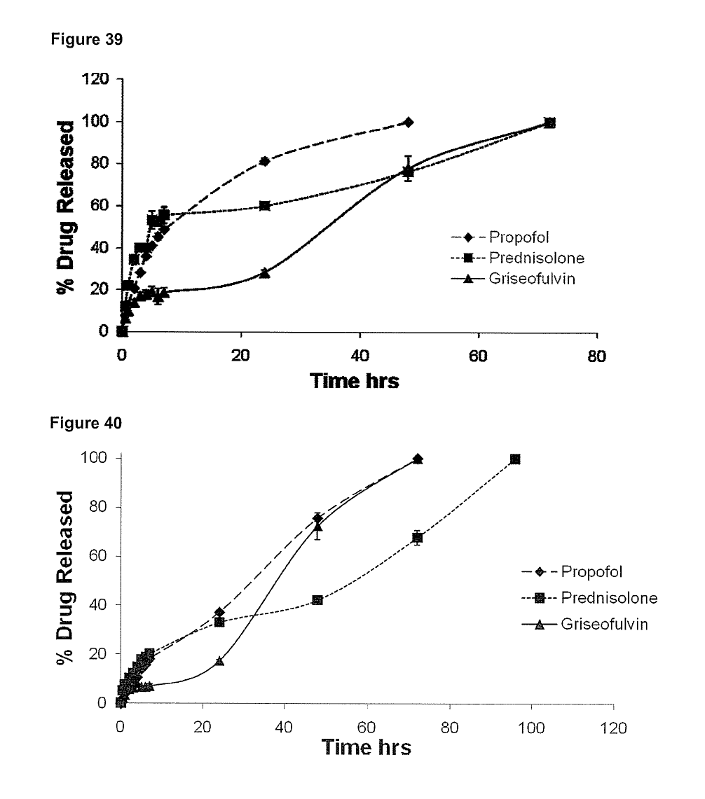

FIG. 39 shows an in vitro release of propofol, prednisolone and griseofulvin from Ch₅ formulations in PBS at 37° C.;

FIG. 40 shows an in vitro release of propofol, prednisolone and griseofulvin from Dansyl₁₀ formulations in PBS at 37° C.;

FIG. 41 shows the percentage of drug lost from nano aggregates of Ch₅ over 4 wks;

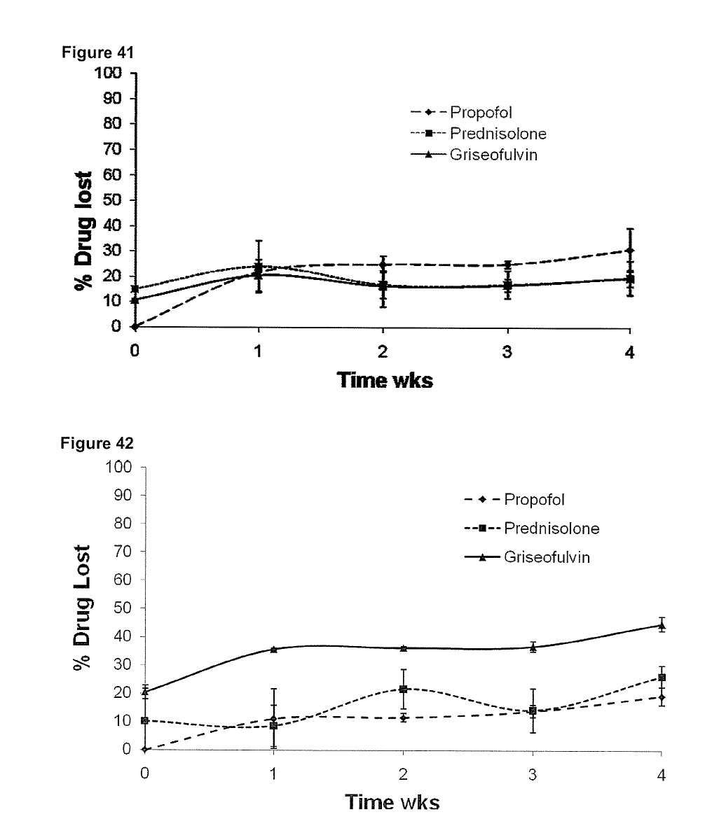

FIG. 42 shows the percentage of drug lost from nano aggregates of Dansyl₁₀ over 4 wks;

FIGS. 43A and B show the percentage Haemolytic effect of A) Fmoc₅, Fmoc₁₀, Dansyl₅ and Dansyl₁₀, B) Ch₅ on bovine red blood cells; and

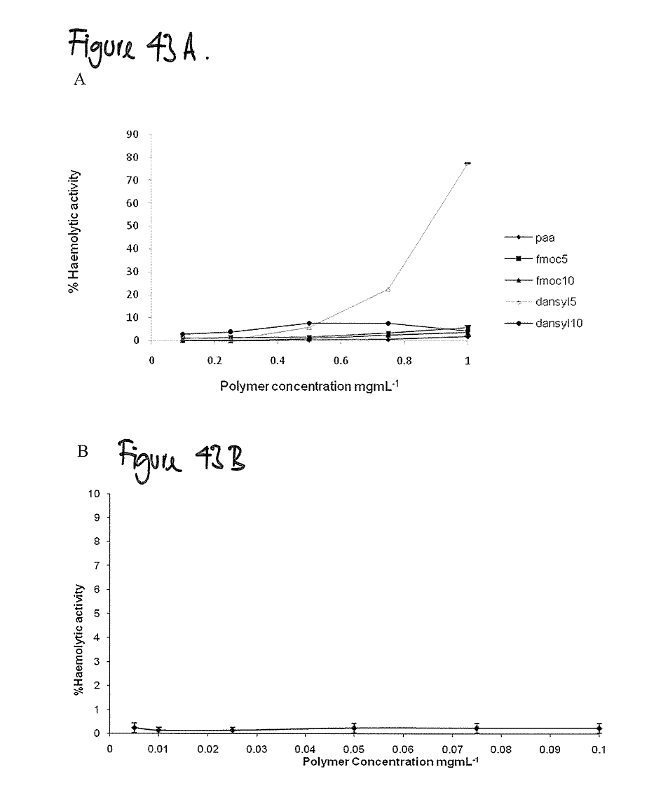

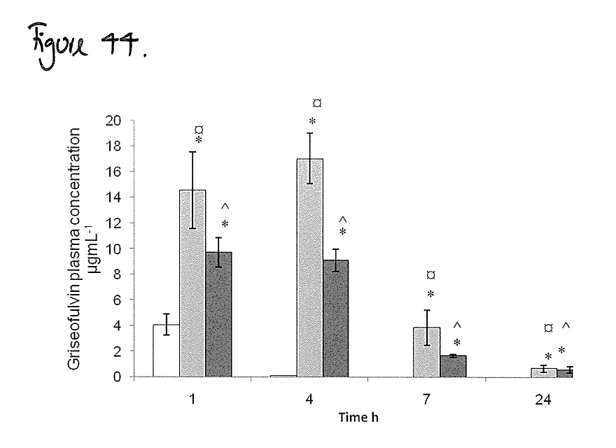

FIG. 44 shows Mean plasma griseofulvin concentration (μgmL⁻¹) following administration of griseofulvin by oral gavage to male Sprague dalwey rats under fasted conditions over 24 h, griseofulvin in water (n=4), Ch₅, griseofulvin (n=3, ave) and Dansyl₁₀, griseofulvin (n=4). Error bars=SD. *p<0.001 formulations vs. griseofulvin in water, ¤p,0.001 Dansyl₁₀, griseofulvin vs. Ch₅, griseofulvin, and ̂p,0.001 Ch₅, griseofulvin nvs. Dansyl₁₀, griseofulvin.

The invention will now be further described by way of the following illustrative examples.

### Polymer Synthesis

### Synthesis of PAA.

10 g of 15 kDa PAA.HCl was dissolved in distilled water and approximately 8 g of NaOH pellets were added until an alkaline pH (˜13) was reached. The solution was then subjected to exhaustive dialysis against 5 L of distilled water for 24 hr using 7000 Dalton membrane with at least six water changes at two hour intervals for the first eight hours. The dialysate was then freeze-dried. The freeze-dried material consists of PAA free base (as shown in FIG. 1).

This PAA free base will then be used as the starting polymer for all amphiphilic polymer synthesis.

Synthesis of 5-Dimethylamino-1-naphthalenesulfonyl-PAA (Dansyl-PAA).

Purified PAA (2 g) was dissolved in 100 ml of a dioxane and water mixture having a dioxane:water ratio of 1:1 v/v. Sodium carbonate was added (5% mole—0.1855 g, 10% mole—0.371 g) and the mixture stirred until homogeneity was achieved. Dansyl chloride 5% (mole—0.472 g, 10% mole—0.944 g) was dissolved in dioxane (20 ml). The mixture was then added drop wise to the polymer solution over 2 hr at 0° C., the reaction was then stirred for an additional 4 hr at 0° C. and 8 hr at room temperature (as shown in FIG. 2). The solvents were removed using a rotary film evaporator; the residue was washed with diethyl ether. The dry residue was dissolved in deionised water and dialysed against water for 24 hr using 12-14000 Dalton membrane. The solution was placed in the freeze drier for 48 hr and the product was recovered. The Dansyl-PAA had a yellow cotton-like appearance. 1.9 g of (Dansyl-PAA) was synthesised (FIG. 3b).

Synthesis of 9-Fluorenylmethoxy carbonyl-PAA (Fmoc-PAA).

Purified PAA (2 g) was dissolved in 100 ml of a dioxane and water mixture having a dioxane:water ratio of 1:1 v/v. Sodium carbonate was added (5% mole—0.1855 g, 10% mole—0.371 g) and the mixture stirred until homogeneity was achieved. Fmoc chloride (5% mole—0.4527 g, 10% mole—0.9054 g) was dissolved in dioxane (20 ml). The mixture was then added drop wise to the polymer solution over 2 hr at 0° C., the reaction was then stirred for an additional 4 hr at 0° C. and 8 hr at room temperature (as shown in FIG. 2). The solvents were removed using a rotary film evaporator; the residue was washed with diethyl ether. The dry residue was dissolved in deionised water and dialysed against water for 24 hr using 12-14000 Dalton membrane. The solution was placed in the freeze drier for 48 hr and the product was recovered. The Fmoc-PAA had a white cotton-like solid appearance. 2.1 g of (Fmoc₅-PAA) was synthesised (FIG. 3c).

### Synthesis of Naphthalene-PAA (Naphth-PAA).

Purified PAA (2 g) was dissolved in 100 ml of a dioxane and water mixture having a dioxane:water ratio of 1:1 v/v. Sodium carbonate was added (5% mole—0.1855 g, 10% mole—0.371 g) and the mixture stirred until homogeneity was achieved. 1-Naphtholyl chloride (5% mole—238 μl, 10% mole—476 μl) was dissolved in dioxane (20 ml). The mixture was then added drop wise to the polymer solution over 2 hr at 0° C., the reaction was then stirred for an additional 4 hr at 0° C. and 8 hr at room temperature (as shown in FIG. 2). The solvents were removed using a rotary film evaporator; the residue was washed with diethyl ether. The dry residue was dissolved in deionised water and dialysed against water for 24 hr using 12-14000 Dalton membrane. The solution was placed in the freeze drier for 48 hr and the product was recovered. The Naphth-PAA had a white cotton like solid appearance. 1.8 g of (Naphth-PAA) was synthesised (FIG. 3d).

### Characterisation of Polymers

### Elemental Analysis

The C, H and N, Cl and S(Dansyl-PAA only) elemental analysis was carried out using a Perkin Elmer 2400 instrument.

The polymer (20 mg) was combusted in a flask containing pure O₂, H₂O₂ and KOH for 0.5 hrs. The flask was then filled with distilled water and cooled back down to room temperature. Ethanol was added before acidification using HNO₃. The final solution was titrated against mercuric nitrate with diphenyl carbazone indicator. The percentage of halogen was determined relative to sample weight.

All the polymers were successfully synthesised and the mole % substitution of the hydrophobic pendant groups (Cholesterol, Fmoc, Dansyl and Napthalene were determined by elemental analysis (Table 1).

TABLE 1
Elemental analysis results of hydrophobic and hydrophilic
modified polymers.

| | % Mole Substitution |
|---|---|
| Modified Polymer | from Elemental Analysis |
| Ch₅-PAA | 5.03 |
| Fmoc₅-PAA | 4.70 |
| Dansyl₅-PAA | 5.60 |
| Naphth₅-PAA | 5.50 |

### ¹H NMR

¹H NMR analysis was carried out on the modified polymers in deuterated methanol (MeOD) using a 400 MHz Bruker Spectrometer. All samples were run at 25° C.

FIG. 4 shows the ¹H NMR spectra of the PAA backbone, Ch-PAA and Dansyl-PAA. The resonance peaks at 1 and 1.5 ppm have been assigned to the CH₂ and CH groups on the chain of the polymer backbone. The resonance peak at 2.5 ppm is due to the CH₂ adjacent to the amino group, the latter causing a downfield shift in the spectrum. In the Ch-PAA spectrum additional peaks at 3 ppm are assigned to the H groups on the cholesterol moiety. The presence of Dansyl functionality to the backbone produced extra peaks at 3 ppm. More importantly, peaks occurring between 7-8 ppm, are due to the hydrogens of the aromatic rings on the dansyl group, thus confirming the successful synthesis of the Dansyl-PAA. The Fmoc-PAA and Naphth-PAA spectra (not shown) also showed the presence of the aromatic ring peaks between 7-8 ppm.

NMR spectra were used to confirm the presence of the hydrophobic pendant side chains in the modified polymers.

### FTIR

FTIR analysis was carried out using a Perkin Elmer, Spectrum, BX, UK, with a diamond powder tip attachment. The polymers were placed under the diamond tip and 20 scans were run for each sample.

FIG. 5 shows the FTIR spectra of PAA, Ch-PAA and Dansyl-PAA. The FTIR spectra for PAA show the presence of two peaks at 2800 cm⁻¹ and 1382 cm⁻¹. These peaks were due respectively to the C—H bond stretching and bending in the polymer backbone. A broad water peak was observed in all the samples above 3000 cm⁻¹ (O—H bond stretch), this was due to the hydroscopic nature of the polymers. The inverted peak at 2200 cm⁻¹ was assigned as the carbon dioxide peak in relation to the background sample. Absorbance occurring at 1500 cm⁻¹ is due to the C—C bond stretching and bending of the polymer backbone.

The IR spectrum for Ch-PAA should show an extra peak at 1600 cm⁻¹ due to the C═O bond stretching of the carbonyl group, although this is not witnessed as the peak is masked. The spectra looks similar to the PAA backbone alone however, the IR spectra for the Ch-PAA appears more intense. This would suggest the presence of additional C—H bonds within the molecule arising from the addition of the cholesterol moiety. The peak present in the fingerprint region (800 cm⁻¹) of Dansyl-PAA is characteristically due to the C—N bond stretching and bending of the dansyl moiety. The C—N bond stretching also gives rise to a peak at around 3000 cm⁻¹, however, this peak has been masked by the broad O—H bond stretch. The presence of the peak at 800 cm⁻¹ indicates the presence of the dansyl moiety. The spectra for the Fmoc-PAA and Naphth-PAA show additional peaks at 700-750 cm⁻¹ in the fingerprint region arising from the presence of aromatic moieties in the compound, thus suggesting the synthesis was successful.

### Characterisation of Nano-Aggregates

### Sizing of Nano-Aggregates

Polymeric self-assemblies were formed by probe sonication of the polymer in water before particle size measurement was carried out using a photon correlation spectrometer (Zetasizer Nano-DS, Malvern Instruments, UK). All measurements were conducted in triplicate at 25° C. and an average value was determined.

The size of the polymeric self-assemblies formed in aqueous solution ranged from 120 nm (Dansyl-PAA) to 199 nm (Fmoc-PAA), as shown in Table 2. The polydispersity index obtained for all samples was below 0.29, indicating mostly uniform peaks were being produced and that all the self-assemblies formed were of a narrow size distribution.

TABLE 2
Results form size analysis of modified polymers.

| Modified Polymer | Size (nm) | Polydispersity Index |
|---|---|---|
| Ch-PAA | 183 | 0.167 |
| Fmoc-PAA | 199 | 0.140 |
| Dansyl-PAA | 120 | 0.285 |
| Naphth-PAA | 188 | 0.127 |

UV-Visible Spectrophotometry

A stock solution of methyl orange (25 μM) was prepared with sodium tetraborate buffer (0.02M, pH 9.4) in deionised water. The solution was placed in a sonic bath for 3 hrs. Concentrations of modified polymer (0.00145-3 mgmL⁻¹) were made up using the methyl orange as the diluent. Each sample was sonicated for 5 mins and allowed to cool to room temperature. The polymer solutions of varying concentration (0.00145-3 mgmL⁻¹) were placed in the UV-visible spectrophotometer (Agilent 8453) and their maximum absorbance was recorded (350-600 nm). A methyl orange stock solution (25 μM) was used according to Uchegbu's method.

A hydrophobic probe such as methyl orange can be used to study the presence of surfactant hydrophobic domains in aqueous solution by monitoring the hypsochromic shift in the methyl orange absorption spectra. Methyl orange has a λₘₐₓ at 464 nm in the presence of UV light. When a surfactant is diluted in a methyl orange solution, a hypsochromic shift is experienced on the UV spectra of the surfactant. This is due to the methyl orange favouring the hydrophobic core formed from the micelles of the surfactant.

FIG. 6 shows the effect of the concentration of Ch₅-PAA on the peak absorbance of methyl orange (0.00145-3 mgmL⁻¹) at 464 nm. Methyl orange studies were carried out in order to observe a hypsochromic shift at the Critical Aggregation Concentration (CAC) value of the modified polymer. A shift was observed at 0.3 mgmL⁻¹ for Ch-PAA.

FIG. 7 shows the effect of the concentration of Fmoc-PAA on the peak absorbance of methyl orange at 464 nm. Methyl orange studies were carried out in order to observe a hypsochromic shift at the CAC value of the modified polymer. A shift was observed at 2 mgmL⁻¹ for Fmoc-PAA.

All the other polymer conjugates (Dansyl-PAA and Naphth-PAA) tested did not show any significant shift and this may be due to the method being insensitive or due to the presence of the naphthalene hydrophobic moiety in dansyl and naphthalene PAA polymers (normally used as fluorophores) interfering with or quenching the methyl orange absorbance.

### Surface Tension

The polymers were made up in aqueous solution (0.00145-3 mgmL⁻¹) and sonicated for 5 mins before cooling to room temperature. The surface tension of the polymer solutions was measured at 25° C. using a torsion balance (OS, White Electrical Instrument Co, London). The platinum ring and platform were cleaned with 98% ethanol and deionised water. The measurement was conducted in triplicate for each polymer solution to obtain an average value. The surface tension of deionised water was determined between each concentration to ensure no cross-contamination of samples had occurred.

When amphiphilic molecules such as surfactants accumulate at a liquid interface, their hydrophobic moiety protrudes out the surface layer into the gaseous phase above. The intrusion of the surface layer results in the replacement of some water molecules with non-polar groups such as hydrocarbons. The attractive forces between water molecules and non-polar groups is significantly less than that of water-water interactions and therefore an expansion of the interface occurs. Subsequently the surface tension is reduced in the presence of surfactants. The surface tension is decreased with addition of very low concentrations of surfactant. The surface tension also decreases as the concentration of the amphiphile is increased.

When the interface of the aqueous environment is ‘saturated’ with surfactant molecules, addition of further surfactant does not result in the decline of surface tension. The remaining molecules in solution find other means of shielding their hydrophobic group from the aqueous environment and self-assemble into aggregates known as micelles. The Critical Micellar Concentration (CMC) value can therefore be determined from a surface tension plot.

FIG. 8 shows the surface tension results for 5% mole substituted with cholesteryl, fmoc, dansyl and naphthalene modified polymers.

PAA substituted with different 5% mole hydrophobic groups exhibits different trends in the surface tension indicating the hydrophobic groups have a major impact on the aggregations of the amphiphiles in the aqueous solution, as shown in FIG. 8. The appearance of the surface tension graphs changed dramatically depending on the type of hydrophobic group present. The Ch-PAA showed to have a clear CAC value at 0.093 mgmL⁻¹ and the Dansyl-PAA at 0.5 mgmL⁻¹. However an interesting observation was made upon determination of the Fmoc-PAA and the Naphth-PAA. The graph was found to have two points of inflection, suggesting the presence of two CAC values. This is most unusual and thus far has not been reported elsewhere.

A possible explanation could be due the phenomenon of excimers. An excimer is a short-lived dimeric molecule formed from two species, at least one of which is in an excited electronic state. They are often diatomic and are formed between two atoms or molecules that would not bond if they were both in their ground states.

The first CAC value observed is as a result of intramolecular aggregation as the polymer strands come together to shield the hydrophobic groups from the aqueous environment. The second CAC value observed is thought to be due to excimer formation occurring as more polymer strands are coming closer together and stacking of the molecules begins. The CAC values were 0.4 and 1.5 mgmL⁻¹ and 0.19 and 0.5 mgmL⁻¹ for Fmoc-PAA and Naphth-PAA respectively.

The phenomenon observed was unique to these two polymers as they possess a planar stereochemistry, thus tight stacking could occur at higher concentrations. However, the cholesterol moiety was too bulky and lacks planarity for such stacking to occur. The dansyl molecule, on first inspection, looks similar to the Fmoc and the Naphth moieties, however the presence of sulphur-oxygen double bonds may give a 3D-structure that could hinder any stacking.

### Fluorescent Spectra of Fmoc-PAA

To further investigate the theory of excimer formation at higher concentrations of Fmoc-PAA, a fluorescent spectra was run over a range of concentrations (0.023, 0.375 and 3 mgmL⁻¹) (as shown in FIG. 9). Fmoc-PAA was dissolved in deionised water and sonicated for 10 mins. After cooling to room temperature samples of 0.023-3 mgmL⁻¹ concentration were run on the Luminescence Spectrometer (Perkin Elmer LS55) with excitation wavelength set at 259 nm. The samples were scanned between 200-500 nm at 400 nm sec⁻¹, excitation slit 15 nm and emission slit 3 nm producing an emission spectra.

FIG. 9 shows a fluorescent spectra of Fmoc-PAA (0.023-3 mgmL⁻¹) scanned between 200-900 nm using a Perkin Elmer LS55 Luminescence spectrometer. Excitation 259 nm, excitation slit 15 nm, emission slit 3 nm, scan speed 400 nm sec⁻¹.

The resultant spectra clearly demonstrated that as the concentration is increased, the emission peak shifts from 315 nm across a range of wavelengths and the intensity increases to a maxima at 560 nm. However at the highest concentration (3 mgmL⁻¹) the emission appears to be sequestered. The spectra produced gives a good indication that excimer formation has occurred as peak shift is a common occurrence. The spectral changes upon excimer formation show the appearance of a new, broader shifted band in the emission spectra. This is due to the charge transfer interaction and stabilisation occurring especially in polar solvents.

### Transmission Electron Microscopy

All samples were examined using Formvar/Carbon-coated 200 mesh nickel grids. The grids were glow discharged and the specimens in distilled water were dried down with filter paper to a thin layer onto the hydrophilic support film.

A 1% aqueous methylamine vanadate (20 μl) (Nanovan; Nanoprobes, Stony Brook, N.Y., USA) stain was applied and the mixture dried down immediately with filter paper to remove excess liquid. The dried specimens were imaged with a LEO 912 energy filtering transmission electron microscope at 120 kV. Contrast enhanced, zero-loss energy filtered digital images were recorded with a 14 bit/2K Proscan CCD camera.

FIG. 10 shows the TEM analysis of Ch-PAA (1 mgmL⁻¹). FIG. 11 shows the TEM analysis of Fmoc-PAA at two concentrations (a) 0.4 mgmL⁻¹ and (b) 2 mgmL⁻¹.

The Ch-PAA TEM (as shown in FIG. 10) and Fmoc-PAA (as shown in FIGS. 11a and b) both show the self-assemblies formed in aqueous solution as colloidal dispersions. The Ch-PAA micrograph showed the nano particles to be less than 100 nm in size.

The size estimation from the micrograph was lower that the size found using PCS (183 nm). This is due to the fact that the PCS measures the hydration layer around the particles and this gives rise to the larger size measured. The preparation method for TEM analysis does not encourage aggregation of the nanoparticles and thus the values are smaller. This method therefore may give more accurate size estimation.

The Fmoc₅-PAA TEM (as shown in FIGS. 11a and b) showed that self-assemblies formed at both the lower (0.4 mgmL⁻¹) and the higher (2 mgmL⁻¹) concentrations. The concentrations tested were significant due to the two CAC values observed from the surface tension measurement. The self-assemblies at the higher concentration appear to be larger (50 nm) compared to those in the lower concentration sample (25 nm). This results from a larger number of polymer chains aggregating together and therefore increasing the self-assembly size.

The TEM of the 0.4 mgmL⁻¹ sample confirms that self-assemblies are formed at this lower concentration and therefore the first CAC value obtained from the surface tension measurement is valid.

### Polymer Aggregation Models

The results from this work indicate that changing the hydrophobic group on the PAA backbone has great impact on the properties of the self-assemblies formed in aqueous solution. The size varied little between the modified polymers formed. However the greatest factor affected was the CAC value. This was apparent mostly when measuring the surface tension measurements. The two flexible aromatic groups (Fmoc and naphthalene), showed two CAC values, whereas the Cholesterol and Dansyl groups only showed one value. This may be due to steric hindrance of the molecules and their inability to stack on top of each other. Interestingly this suggested that the flexible aromatic molecules and the bulkier molecules formed self-assemblies in the aqueous environment in two very different manners. FIG. 12 shows the polymer aggregation model for Cholesterol-PAA and Dansyl-PAA.

The CAC values obtained for the Ch-PAA polymer differed when using the methyl orange probe (0.375 mgmL⁻¹) and the surface tension measurement (0.093 mgmL⁻¹). This showed the varying sensitivity levels of the methods of determining the CAC values. The surface tension graph for the Ch-PAA and for the Dansyl-PAA showed the presence of only one CAC value (0.093 and 0.5 mgmL⁻¹ respectively). Below the CAC values, no self-assemblies formed, above the CAC values, the surface tension measurement remained constant, even at concentrations of up to 3 mgmL⁻¹. This suggested that only one aggregation mechanism was being employed, intermolecular aggregation where more than one polymer strand was coming together to shield their hydrophobic groups in a larger self assembly, as shown in FIG. 12.

The TEM of the Fmoc-PAA (FIG. 11) showed that at lower concentrations of 0.4 mgmL⁻¹ self-assemblies where formed, this agreed with the CAC prediction form the surface tension graph (FIG. 8). This was resulting from intramolecular aggregation of the polymer, whereby one single polymer strand was aggregating with itself to form a self assembly (as shown in FIG. 13). This was apparent from the small size of the micelles formed. At higher concentrations of Fmoc-PAA, the self-assemblies had increased in size and formed a colloidal dispersion. This was due to the polymer intermolecular aggregation. It was at the stage of intermolecular aggregation that excimer stacking of the hydrophobic pendants occurred, making the self-assemblies more stable. This was confirmed with the shift of emission on the fluorescent spectra.

Novel comb shaped amphiphilic polymers have been successfully synthesised which are able to form nano self-assemblies in aqueous solution. The results showed that the presence of different hydrophobic groups gave rise to significant differences in properties both with the polymer and their self assemblies. This was particularly apparent in the surface tension measurements where possible excimer formation was occurring with the planar stacking of the Fmoc and Naphthalene molecules. The steric hindrance however of the Cholesterol and Dansyl molecules prevented any stacking from occurring.

### Safety Profile

The safety profile of various drugs and nano-aggregates of the drugs and polymers were investigated using the standard colorimetric MTT (3-(4,5-Dimethylthiazol-2-yl)-2,5-diphenyltetrazolium bromide) assay. Table 3 shows the formulation that includes the Dansyl₁₀-PAA and hydrophobic drugs such as etoposide and BNIPDacot showed significant increase in cytotoxicity in Human Embryonic Kidney (HEK) 293 cells by a factor of ten and twenty-nine respectively.

TABLE 3
MTT assay of polymers and formulations on HEK 293 cells
(n = 3 ± SD), IC₅₀ of the polymer was used in the formulations

| | | Increase |
|---|---|---|
| Sample | IC₅₀ mgmL⁻¹ (n = 3 ± SD) | in toxicity |
| PAA | 0.01624 (0.00126) | — |
| Etoposide | 0.00248 (0.00041) | — |
| BNIPDaoct | 0.00496 (0.00005) | — |
| Ch₅-PAA | 0.05135 (0.0045) | — |
| Ch₅-PAA + Etoposide | 0.00135 (0.00116) | 1.8 fold |
| Ch₅-PAA + BNIPDaoct | 0.00016 (0.00046) | 31 fold |
| Dansyl₁₀-PAA | 0.01934 (0.00186) | — |
| Dansyl₁₀-PAA + Etoposide | 0.00024 (0.00038) | 10 fold |
| Dansyl₁₀-PAA + BNIPDaoct | 0.00017 (0.00008) | 29 fold |

The MTT assay using HEK cells showed that both polymers enhanced the activity and cytotoxic effect of both etoposide and BNIPDacot anticancer drugs up to 31-fold.

The haemolysis assay, FIG. 14, showed no significant haemolytic activity (<2%) at the concentration tested (0.005-1 mgml⁻¹) for both Ch₅-PAA and Dansyl₁₀-PAA, indicating the good haemocompatability of the polymers. The haemolysis assay gives information on the ability of the PAA polymers to cause the release of haemoglobin on exposure to red blood cells. FIG. 14 shows none of the polymers tested (0.1 mg/ml) cause haemolysis since all percentages obtained are below 25%.

### Biological Characterisation

### Haemolysis Assay

Fresh bovine blood was washed with phosphate buffered saline (PBS buffer) (0.1 M) and centrifuged (2500 rpm) for 10 min at 4° C. The supernatant was discarded. This process was repeated until the supernatant was clear. The erythrocyte was isolated and weighed and a 3% (w/v) dilution in PBS solution was carried out. The red blood cell solution (80 μL) was pipetted into the wells of a 96 well round bottom plate.

A 10 mgmL⁻¹ stock solution of polymer was made up in deionised water and pH adjusted to pH 7.4 with NaOH/HCl. Various concentrations (1-0.05 mgmL⁻¹) of polymer solution were prepared from the stock solution using PBS as the diluent. The wells of the plate were then treated with 80 μl of increasing polymer concentrations. PBS and Triton X (80 μL each) were used as the negative and positive controls respectively. The plates were incubated at 37° C. for 4 h before being centrifuged at 2500 rpm for 10 min at 4° C. The supernatant (100 μL) was transferred to a flat bottomed 96 well plate for analysis. The absorbance of the plate was read at 570 nm on a microplate reader (Ascend Lab-Systems, UK). The % haemolysis was calculated in relation to the positive and negative controls. The remaining pellets were viewed under the light microscope (Leica DM3000B, Leica UK) and observations were recorded.

### Cytotoxicity Assay

### Cytotoxicity of Modified Polymers

The cytotoxicity of the modified PAA polymers was determined using the MTT assay model. Caco-2 cells were cultured in minimum essential medium (MEM) containing 10% foetal bovine serum (FBS), 1% L-glutamine and 1% non essential amino acids (NEAA).

A 10 mgmL⁻¹ polymer solution was prepared using sterile water as the diluent. The solution was further diluted with media to form a 0.5 mgmL⁻¹ stock solution. From the stock solution nine dilutions (0.2-1×10⁻⁴ mgmL⁻¹) were made using media as the diluent.

Caco-2 cells (200 μL, 10000 cells/well) in exponential growth phases were seeded into a 96-well flat bottomed plate and incubated for 24 h at 37° C. with 5% CO₂. The media was replaced with increasing polymer concentrations (0.5-1×10⁻⁴ mgmL⁻¹) as prepared above. Media and Triton X (1:5 PBS) were the negative and positive controls respectively. The plate was incubated as before. After 24 h the polymer solutions were removed and replaced with fresh media. The plate was further incubated for 24 h before the media was removed and replaced once more. 3-[4,5-dimethylthiazol-2-yl]-2,5-diphenyltetrazolium (MTT) (50 μL, 5 mgmL⁻¹) was added to the wells and the plate was incubated (37° C. with 5% CO₂) in the dark for 4 h. After this time the MTT solution was removed from the wells. The remaining purple formazan complexes were dissolved in DMSO (200 μL) and L-glycine buffer (25 mL, made up of 3.75 g glycine and 2.93 g NaCl made up in 500 mL and pH adjusted to pH 10.5) and the absorbance of the plates was read at 570 nm using a microplate reader (Ascend Lab-Systems, UK). Percentage cell viability was calculated relative to the positive and negative controls.

### Cytotoxicity of Anticancer Drug Formulations

An MTT assay was carried out using Caco-2 and HEK cells. Caco-2 cells were cultured as previously described. HEK cells were cultured in Dulbecco's minimum essential medium (DMEM) containing 10% FBS and 1% penicillin streptomycin (Penstrep). Cytotoxicity assays for anticancer drug etoposide (0.5-1×10⁻⁶ mgmL⁻¹) and formulation of the anticancer drug using Ch₅ and Dansyl₁₀ polymers were carried out. The polymers were fixed at a concentration of 0.005 mgmL⁻¹ were the cell viability is 90% (IC90) based on the MTT assay.

Polymeric self-assemblies were formed by probe sonicating the polymers in sterile water (5 mgmL⁻¹). The polymer stock solutions were diluted to 0.005 mgmL⁻¹ with media. Etoposide stock solution (20 mgmL⁻¹) was prepared by diluting the drug in DMSO. The formulations were prepared by addition of drug into 0.005 mgmL⁻¹ polymer solution.

### Biological Characterisation

### Haemolysis Assay

Haemolysis assay was carried out between 0.1-1 mgmL⁻¹ for all polymers (FIG. 43A) except Ch₅. This was due to the Ch₅ precipitating out of solution on addition of PBS buffer. Therefore the haemolysis was carried out at lower concentrations of 0.05-0.1 mgmL⁻¹ (FIG. 43B) where precipitation was not evident.

Introduction of hydrophobic groups onto the PAA backbone increased the haemolytic effect of the PAA (FIG. 43A-B). However, most of the amphiphiles caused no haemolytic effect on the red blood cells with (% haemolysis≦10% from polymer concentration from 0.05 to 1 mgmL⁻¹) with exception to Dansyl₅. The haemolytic activity varied with different hydrophobic pendant group on the amphiphiles. The Dansyl₁₀ polymers resulted in notably higher haemolytic activity than the other groups. As a general trend, higher levels of hydrophobic grafting lowers the haemolytic activity. The reduction in haemolytic activity results in greater biocompatibility. Most of the PAA amphiphiles showed an independent haemolytic trend, whereby, increase in polymer concentration did not have an effect on haemolytic activity. However, Dansyl₅ appeared to be concentration dependant (FIG. 43A). This possibly explained their unusually high haemolytic activity at the higher concentration ranges. The red blood cells where imaged under the light microscope to identify any changes in the appearance of red blood cells. The morphology of the red blood cells did not change in the presence of the 5 or 10% hydrophobic grafted polymers. The type of hydrophobic group and level of grafting did not appear to alter or disrupt the red blood cell structure. However, at higher concentrations of Dansyl₅ cell lysis occurred resulting in reduced population of blood cells.

### Cytotoxicity Assay

The MTT assay was carried out to determine the polymer concentration at which only 50% (IC50) of the cells population were viable. Polymers with lower IC50 values, have a greater cytotoxic effect. No notable difference was observed between the IC50 of the modified polymer and the unmodified PAA backbone (Table 20). The different hydrophobic groups had only a slight impact on the IC50 values. The IC50 of the unmodified PAA backbone was 23.3 μgmL⁻¹ and the presence of the hydrophobic pendants did not significantly change the IC50 value of the backbone. Ch₅ had a higher 1050 value (37.4 μgmL⁻¹) indicating that the presence of the cholesteryl group improved the safety profile of the polymer. The presence of Dansyl had the adverse effect reducing the 1050 to 17.4 μgmL⁻¹ (Dansyl₅). The degree of grafting also had an impact on the cytotoxicity of the polymers. At higher levels of grafting a decrease in cytotoxicity was observed. Fmoc₁₀ and Dansyl₁₀ appeared safer than their 5% counterparts (27.9 and 24.6 μgmL⁻¹ respectively), this perhaps was due to the reduced number of primary amine groups present, thus reducing the cytotoxicity of the polymer.

TABLE 20
IC₅₀ values for Caco-2 cells treated with modified PAA
polymers (1 × 10⁻⁴-0.5 mgmL⁻¹) for 24 h, n = 3, ave (SD).

| | | Number of fold more |
|---|---|---|
| | IC₅₀ value on Caco-2 cells | cytotoxic compared to IC₅₀ of |
| Polymer | (μgmL⁻¹) | PAA |
| PAA | 23.3 (20.1) | — |
| Ch₅ | 37.4 (3.7) | −1.61 |
| Fmoc₅ | 22.9 (3.61) | 0.98 |
| Fmoc₁₀ | 27.9 (9.62) | −1.19 |
| Dansyl₅ | 17.4 (3.5) | 1.39 |
| Dansyl₁₀ | 24.6 (0.9) | −1.06 |

To determine whether our formulation enhanced the therapeutic effect of the anticancer agent, they were formulated at the polymer IC90 concentration (the concentration at which 90% of cells where viable determined via MTT assay). It is assumed that they have negligible cytotoxic effect on cells, and any change in IC50 value when treated with anticancer formulations when compared to the free drug is as a result of increased uptake of the drug or due to the enhanced therapeutic effect. When Caco-2 and HEK293 cells were treated with etoposide, it appeared that the HEK293 cells had a much lower IC50 values than the Caco-2 cells, 2.5 μgmL⁻¹ and 189.9 μgmL⁻¹ respectively, indicating etoposide is more cytotoxic on HEK cells (Table 21). Perhaps the slower rate of proliferation on healthy HEK cells compared to the carcinoma Caco-2 cells made them more susceptible to the cytotoxic effect in vitro.

TABLE 21
MTT assay of polymers and formulations on Caco-2 and
HEK 293 cells.

| | | | IC₅₀ on | |
|---|---|---|---|---|
| | | | HEK | |
| Polymer/Drug/ | IC₅₀ on Caco-2 | Increase in | 293 cells | Increase in |
| Formulation | cells (μgmL⁻¹) | cytotoxicity | (μgmL⁻¹) | cytotoxicity |
| Ch₅ | 37.4 | — | 51.4 | — |
| Dansyl₁₀ | 24.6 | — | 19.3 | — |
| Etoposide | 189.9 | — | 2.5 | — |
| Ch₅, etoposide | 86.3 | 2.2-fold | 1.4 | 1.8-fold |
| Dansyl₁₀, | 13.6 | 14-fold | 0.2 | 10-fold |
| etoposide | | | | |

IC₉₀ of the polymer was used in the formulations (5 μgmL⁻¹).

When Ch₅, etoposide formulation was exposed to the two cell lines, an equal increase in cytotoxicity was experienced by both cell lines. This indicates that Ch₅ was able to enhance the therapeutic effect of the drug compared with the free etoposide. A similar effect was observed for the Dansyl₁₀, etoposide formulation. However the Dansyl₁₀, etoposide formulation achieved the largest decrease in IC50 values for both cell lines (14-fold and 10-fold respectively), suggesting that the Dansyl₁₀ was a more effective delivery vehicle for the delivery of etoposide to Caco-2 and HEK cells.

### Model Hydrophobic Drugs

The model hydrophobic drugs Propofol (2,6-diisopropylphenol), Prednisolone (1-dehydrocortisone), Griseofulvin ((2S)-trans-7-chloro-2′,4,6-trimethoxy 6′-methylspiro(benzofuran-2[3H],1′-[2]cyclohexene)3,4′-dione), Etoposide (VP-16-213, 4′-Demethylepipodophyllotoxin 9-(4,6-O-ethylidene-β-D-glucopyranoside) and BNIPDaoct (Bisnahthalimidopropyldiaminooctane) were used.

Propofol (FIG. 15) is a commonly used short acting anaesthetic agent with favourable pharmacokinetic abilities. Propofol has an aqueous solubility of 100 μgmL⁻¹ at 25° C. Due to its low water solubility it is formulated as oil in water emulsion for intravenous administration (fixed at 10 mgmL⁻¹). However, intravenous administration of the emulsion proves very painful in patients and often has to be preceded by a painkilling injection. Other problems occur with this formulation due to its high lipid concentration and non-strictly aseptic use causing a high number of infections within patients.

Prednisolone (FIG. 16) is a steroid drug prescribed for the treatment of inflammatory conditions for example arthritis, asthma and cluster headaches. Due to prednisolone's very low solubility (215 μgmL⁻¹ at 25° C.) it possesses poor oral bioavailability. Prednisolone is currently administered in coated tablets or more commonly in a suspension formulation fixed at 3 mgmL⁻¹. The large dose concentration needed for therapeutic effect causes an undesirable taste leading to poor patient compliance.

Griseofulvin (FIG. 17) is a lipophilic drug with an aqueous solubility of 30 μgmL⁻¹ at 25° C. The antifungal properties of Griseofulvin are used in both animals and humans for the treatment of dermatophyte infections. The hydrophobic nature of the drug results in poor oral bioavailability. Currently griseofulvin is administered orally as microcrystalline preparations in 500-1000 mgday⁻¹ doses.

Etoposide (FIG. 18) is one of the most efficient antineoplastic chemotherapeutics used today. The drug acts as a toposisomoerase II inhibitor. DNA topoisomerases are enzymes found in the cell nucleus which allow the cell to change arrangement of its DNA by making transient breaks in the DNA strand. The inhibition of topoisomerase II results in the death of cancerous cells. Clinically etoposide is used for treatment of lung, ovarian and testicular cancer. Etoposide has a low aqueous solubility of 148-153 μgmL⁻¹ at 37° C. The drug is currently formulated in a cosolvent formulation for oral or parenteral routes, however, its poor aqueous solubility has reportedly caused precipitation upon dilution in vivo.

BNIPDaoct (FIG. 19) is a novel anticancer agent of the bisnaphthalamidopropyl series. Bis-naphthalimide derivatives have been reported to possess great potential as cytotoxic drugs for the treatment of cancer. However the compounds exhibit poor solubility in aqueous solutions. BNIPDaoct has negligible aqueous solubility and therefore requires harsh solvents such as DMSO to solubilise the drug.

### Drug Loading of Nanoparticles

Polymer solutions of 1 mgmL⁻¹, 3 mgmL⁻¹ and 6 mgmL⁻¹ were made up in deionised water and probe sonicated for 10 mins to ensure homogeneity. The hydrophobic drug was added in 1:1, 5:1 and 10:1 drug:excipient ratios and the drug-polymer solutions were probe sonicated for a further 10 mins to ensure maximum encapsulation was achieved. After cooling to room temperature, the solutions were filtered using 0.45 μm GDX PVDF syringe filters to ensure any excess unencapsulated propofol was removed.

### Determination of Drug Loading Capacity

### Solubilisation of Propofol

The drug loading capacity of the self assemblies was determined using high performance liquid chromatography (HPLC). For propofol a reverse phase Zorbax ODS 250 mm×46 mm×5 μl HPLC column was used with a flow rate of 1 mlmin⁻¹ of the mobile phase (80:20 methanol:water) in an isocratic mode. The column eluent was monitored at 229 nm using a UV Detector. The samples were diluted with mobile phase and 20 μl was injected onto the column, the resultant peak at 7 mins was analysed. The quantification of amount of propofol present in the samples was determined by comparing to a standard calibration carried out previously with propofol dissolved in methanol (3.9-250 μgmL⁻¹).

### Solubilisation of Prednisolone

The HPLC was carried out using a reverse phase Phenomenex C₁₈ 150 mm×4.6 mm×3.5 μm column with a mobile phase of 1 mlmin⁻¹ in an isocratic mode. A UV detector was set at 243 nm. A mobile phase of 36:64 acetonitrile:water was run through the column and 20 μl of sample was injected. The retention of the peak was 3 mins. The quantification of amount of prednisolone present in the samples was determined by comparing to a standard calibration carried out previously with prednisolone dissolved in mobile phase (6.25-25 μgmL⁻¹).

### Solubilisation of Griseofulvin

A reverse phase Phenomenex C₁₈ 250 mm×46 mm×5 μm HPLC column was used with flow rate of 1 mlmin⁻¹ of the mobile phase (45:55 acetonitrile:45 mM potassium dihydrogen phosphate made up in water and pH adjusted to pH3 with orthophosphoric acid) in an isocratic mode. The column eluent was monitored at 293 nm using a UV Detector. The samples were diluted with mobile phase and 20 μl was injected onto the column, the resultant peak at 9.5 mins was analysed. The quantification of amount of griseofulvin present in the samples was determined by comparing to a standard calibration carried out previously with Griseofulvin dissolved in mobile phase (0.625-10 μgmL⁻¹).

### Solubilisation of Etoposide

A reverse phase Zorbax ODS 250 mm×46 mm×5 μl HPLC column was used with flow rate of 1 mlmin⁻¹ of the mobile phase (40:3:57 methanol:acetonitrile:water) in an isocratic mode. The column eluent was monitored at 229 nm using a UV Detector. The samples were diluted with mobile phase and 20 μl was injected onto the column, the resultant peak at 20 mins was analysed. The drug content was determined by comparing to a calibration carried out on etoposide dissolved in methanol (31.3-500 μgmL⁻¹).

### Solubilisation of BNIPDaoct

The BNIPDaoct content in self assemblies was carried out using reverse phase Zorbax ODS 250 mm×46 mm×5 μl HPLC column was used with flow rate of 1 mlmin⁻¹ of the mobile phase (55:45 buffer:acteonitrile) in an isocratic mode. The buffer was made up of 0.4 g octane sulfonic acid and anhydrous sodium acetate made up to 200 ml with deionised water, the solution was pH adjusted to pH4.5. The column eluent was monitored using a fluorescent Detector. The samples were diluted with mobile phase and 20 μl was injected onto the column, the resultant peak at 10 mins was analysed. A calibration was carried out by dissolving BNIPDaoct in DMSO:water (50:50 v/v) (39-625 μgmL⁻¹).

Sizing of Nano-Aggregates

Size measurements were determined for the optimal drug loaded self assembly for each modified polymer:drug solution as previously described.

### Transmission Electron Microscopy

The optimal formulation was viewed under the TEM as previously described.

### FTIR Analysis of Freeze Dried Formulations

The optimal formulation was prepared and freeze dried. The freeze-dried preparation was run on the FTIR with diamond tip and the peaks identified and compared to the spectra obtained for the freeze dried drug and polymer separately as previously described.

In Vitro Drug Release

The formulation (2 ml) was pipetted inside dialysis tubing (12-14000 kDa) and dialysed in sink against PBS (200 ml, 0.2M) at 37° C. with stirring. At various time points 1 ml of the external PBS was extracted and replaced with 1 ml of fresh PBS. The presence of drug in the collected PBS was determined using HPLC

### Stability of Formulations

The optimal formulation for each drug was stored in an air-tight desiccator at room temperature in dark conditions. After set time periods, the formulations were examined by HPLC analysis to identify the amount of drug encapsulated by the polymeric micelles. This study was carried out to see whether over long periods of time, the formulations degraded. The formulation stability testing was carried out both in solution and as freeze-dried formulations which were reconstituted with water before testing.

### Synthesis of BNIPDaoct

BNIPDaoct was successfully synthesised. Characterisation confirmed the pure product was produced. The ¹H, ¹³C and Dept NMR correlated well with published data.

### Drug Loading of Nanoparticles

### Solubilisation of Propofol

All comb shaped polymers (1, 3 and 6 mgmL⁻¹) were loaded with propofol in 1:1, 5:1 and 10:1 drug:polymer ratio. HPLC was used to determine the maximum loading concentration for each polymer. FIG. 20 shows a typical HPLC spectra for propofol. The retention time for this drug was around 7 mins.

From Table 4 it is observed that each of the modified polymers had its own unique ability to solubilise propofol. No general trend was observed with regards to the concentration or drug loading patterns and maximum drug solubilised.

TABLE 4
Drug loading capacity of propofol in modified polymers
determined using reverse phase HPLC for each drug:polymer ratio n = 3,
resulting excipient:drug ratio's and degree of solubility increase from
aqueous solubility of drug.

| | | | Concentration of | | Increase |
|---|---|---|---|---|---|
| | Polymer | | Propofol | Excipient:Drug | from |
| | Concentration | Drug:Polymer | solubilised mgmL⁻¹ ± | Ratio after | aqueous |
| Polymer | mgmL⁻¹ | Ratio | SD | solubilisation | solubility |
| Ch₅ | 1 | 1:1 | 0.063 (0.040) | 15.87 | 0.0 Fold |
| | | 5:1 | 0.353 (0.093) | 2.83 | 3.5 Fold |
| | | 10:1 | 0.590 (0.407) | 1.69 | 5.9 Fold |
| | 3 | 1:1 | 0.693 (0.319) | 4.33 | 6.9 Fold |
| | | 5:1 | 4.837 (1.521) | 0.62 | 48.4 Fold |
| | | 10:1 | 5.963 (1.954) | 0.50 | 59.6 Fold |
| | 6 | 1:1 | 2.203 (1.484) | 2.72 | 22.0 Fold |
| | | 5:1 | 7.357 (1.152) | 0.82 | 73.6 Fold |
| | | 10:1 | 7.823 (2.058) | 0.77 | 78.2 Fold |
| QCh₅ | 1 | 1:1 | 0.073 (0.076) | 13.70 | 0.0 Fold |
| | | 5:1 | 0.200 (0.087) | 5.00 | 2.0 Fold |
| | | 10:1 | 0.673 (0.242) | 1.49 | 6.7 Fold |
| | 3 | 1:1 | 0.073 (0.023) | 41.10 | 0.0 Fold |
| | | 5:1 | 0.860 (0.211) | 3.49 | 8.6 Fold |
| | | 10:1 | 0.330 (0.121) | 9.09 | 3.3 Fold |
| | 6 | 1:1 | 0.427 (0.232) | 14.05 | 4.3 Fold |
| | | 5:1 | 0.480 (0.160) | 12.50 | 4.8 Fold |
| | | 10:1 | 1.047 (0.412) | 5.73 | 10.5 Fold |
| Fmoc₅ | 1 | 1:1 | 0.037 (0.046) | 27.03 | 0.0 Fold |
| | | 5:1 | 0.080 (0.020) | 12.50 | 0.0 Fold |
| | | 10:1 | 0.037 (0.032) | 27.03 | 0.0 Fold |
| | 3 | 1:1 | 0.090 (0.000) | 33.33 | 0.0 Fold |
| | | 5:1 | 0.100 (0.020) | 30.00 | 0.0 Fold |
| | | 10:1 | 0.067 (0.031) | 44.78 | 0.0 Fold |
| | 6 | 1:1 | 0.460 (0.159) | 13.04 | 4.6 Fold |
| | | 5:1 | 0.093 (0.031) | 64.52 | 0.0 Fold |
| | | 10:1 | 0.060 (0.020) | 100.00 | 0.0 Fold |
| Fmoc₁₀ | 1 | 1:1 | 0.020 (0.000) | 50.00 | 0.0 Fold |
| | | 5:1 | 0.197 (0.070) | 5.08 | 2.0 Fold |
| | | 10:1 | 0.207 (0.015) | 4.83 | 2.1 Fold |
| | 3 | 1:1 | 0.113 (0.029) | 26.55 | 1.1 Fold |
| | | 5:1 | 0.283 (0.029) | 10.60 | 2.8 Fold |
| | | 10:1 | 0.160 (0.035) | 18.75 | 1.6 Fold |
| | 6 | 1:1 | 0.690 (0.265) | 8.70 | 6.9 Fold |
| | | 5:1 | 0.393 (0.064) | 15.27 | 3.9 Fold |
| | | 10:1 | 0.313 (0.112) | 19.17 | 3.1 Fold |
| Dansyl₅ | 1 | 1:1 | 0.013 (0.006) | 76.92 | 0.0 Fold |
| | | 5:1 | 0.477 (0.309) | 2.10 | 4.8 Fold |
| | | 10:1 | 0.120 (0.010) | 8.33 | 1.2 Fold |
| | 3 | 1:1 | 0.193 (0.064) | 15.54 | 1.9 Fold |
| | | 5:1 | 2.200 (0.661) | 1.36 | 22.0 Fold |
| | | 10:1 | 0.553 (0.117) | 5.42 | 5.5 Fold |
| | 6 | 1:1 | 1.713 (0.150) | 3.50 | 17.1 Fold |
| | | 5:1 | 5.870 (2.053) | 1.02 | 58.7 Fold |
| | | 10:1 | 2.113 (0.444) | 2.84 | 21.1 Fold |
| QDansyl₅ | 1 | 1:1 | 0.047 (0.031) | 21.28 | 0.0 Fold |
| | | 5:1 | 0.087 (0.012) | 11.49 | 0.0 Fold |
| | | 10:1 | 0.107 (0.023) | 9.35 | 1.1 Fold |
| | 3 | 1:1 | 0.160 (0.087) | 18.75 | 1.6 Fold |
| | | 5:1 | 0.147 (0.012) | 20.41 | 1.5 Fold |
| | | 10:1 | 0.120 (0.020) | 25.00 | 1.2 Fold |
| | 6 | 1:1 | 0.183 (0.045) | 32.79 | 1.8 Fold |
| | | 5:1 | 0.190 (0.056) | 31.59 | 1.9 Fold |
| | | 10:1 | 0.173 (0.012) | 34.68 | 1.7 Fold |
| Dansyl₁₀ | 1 | 1:1 | 0.013 (0.012) | 76.92 | 0.0 Fold |
| | | 5:1 | 0.893 (0.155) | 1.12 | 8.9 Fold |
| | | 10:1 | 2.327 (0.902) | 0.43 | 23.3 Fold |
| | 3 | 1:1 | 0.427 (0.200) | 7.03 | 4.3 Fold |
| | | 5:1 | 5.667 (1.146) | 0.53 | 56.7 Fold |
| | | 10:1 | 8.580 (2.270) | 0.35 | 85.8 Fold |
| | 6 | 1:1 | 1.767 (0.490) | 3.40 | 17.7 Fold |
| | | 5:1 | 17.147 (0.740) | 0.35 | 171.5 Fold |
| | | 10:1 | 22.373 (0.441) | 0.13 | 223.7 Fold |
| QDansyl₁₀ | 1 | 1:1 | 0.020 (0.010) | 50.00 | 0.0 Fold |
| | | 5:1 | 0.167 (0.029) | 5.99 | 1.7 Fold |
| | | 10:1 | 0.140 (0.020) | 7.14 | 1.4 Fold |
| | 3 | 1:1 | 0.140 (0.010) | 21.43 | 1.4 Fold |
| | | 5:1 | 0.310 (0.132) | 9.68 | 3.1 Fold |
| | | 10:1 | 0.360 (0.094) | 8.33 | 3.6 Fold |
| | 6 | 1:1 | 0.383 (0.032) | 15.67 | 3.8 Fold |
| | | 5:1 | 0.363 (0.015) | 16.53 | 3.6 Fold |
| | | 10:1 | 0.380 (0.182) | 15.79 | 3.8 Fold |

The amount of propofol solubilised ranged from 0.19 to 22.4 mgmL⁻¹ (QDansyl₅ and Dansyl₅ respectively). The maximum concentration solubilised by each of the amphiphilic polymers is shown in FIG. 21 along with the aqueous solubility of propofol (0.1 mgmL-1). Cholesterol (6 mgmL⁻¹, 10:1) and Dansyl (6 mgmL⁻¹, 10:1) proved excellent in the solubilisation of propofol; these polymers improved the aqueous solubility 78-fold and 223-fold respectively. These formulations possessed excipient to drug levels as low as 0.77 and 0.13 respectively. However, the quaternised counterparts of all the modified polymers were not as effective with a maximum of 1.05 mgmL⁻¹ propofol solubilised by cholesterol.

The propofol solubilisation study could no be carried out using the Naphth-PAA polymers as their inherent fluorescent nature meant that the polymer was excited around the same wavelength as the propofol and thus interference was observed on the HPLC spectra (FIG. 22).

### Solubilisation of Prednisolone

Solubilisation studies for prednisolone were carried out using the amphiphilic polymers. However, using the previous propofol data the range of polymer was narrowed. QCh₅ was the only hydrophilic polymer studied and no Naphth polymers were tested. The polymers were tested at 1, 3 and 6 mgmL⁻¹ and using 1:1, 5:1 and 10:1 drug:polymer ratio. The retention time for prednisolone was around 3 mins (FIG. 23). Table 5 shows the concentration of prednisolone solubilised at each concentration and drug:polymer ratio. The maximum solubilisation (FIG. 24) achieved ranged from 2.05 to 31.82 mgmL⁻¹ (QCh₅ and Dansyl₁₀ respectively).

TABLE 5
Drug loading capacity of prednisolone in modified polymers
determined using reverse phase HPLC for each drug:polymer ratio n = 3,
resulting excipient:drug ratio's and degree of solubility increase from
aqueous solubility of drug.

| | | | Concentration of | | Increase |
|---|---|---|---|---|---|
| | Polymer | | Prednisolone | Excipient:Drug | from |
| | Concentration | Drug:Polymer | solubilised mgmL⁻¹ ± | Ratio after | aqueous |
| Polymer | mgmL⁻¹ | Ratio | SD | solubilisation | solubility |
| Ch₅ | 1 | 1:1 | 0.969 (0.113) | 1.03 | 4.4 Fold |
| | | 5:1 | 1.183 (0.036) | 0.85 | 5.4 Fold |
| | | 10:1 | 1.396 (0.157) | 0.72 | 6.3 Fold |
| | 3 | 1:1 | 1.417 (0.055) | 2.11 | 6.4 Fold |
| | | 5:1 | 2.517 (0.448) | 1.19 | 11.4 Fold |
| | | 10:1 | 2.024 (0.404) | 1.48 | 9.2 Fold |
| | 6 | 1:1 | 2.175 (0.444) | 2.76 | 9.9 Fold |
| | | 5:1 | 3.424 (0.265) | 1.75 | 15.6 Fold |
| | | 10:1 | 7.045 (0.793) | 0.85 | 32.0 Fold |
| QCh₅ | 1 | 1:1 | 0.310 (0.062) | 3.23 | 1.4 Fold |
| | | 5:1 | 1.747 (0.145) | 1.72 | 7.9 Fold |
| | | 10:1 | 1.639 (0.167) | 0.61 | 7.5 Fold |
| | 3 | 1:1 | 1.698 (0.134) | 1.77 | 7.7 Fold |
| | | 5:1 | 1.636 (0.236) | 1.83 | 7.4 Fold |
| | | 10:1 | 1.627 (0.226) | 1.84 | 7.4 Fold |
| | 6 | 1:1 | 1.757 (0.257) | 3.41 | 8.0 Fold |
| | | 5:1 | 1.845 (0.272) | 3.25 | 8.4 Fold |
| | | 10:1 | 2.047 (0.289) | 2.93 | 9.4 Fold |
| Fmoc₅ | 1 | 1:1 | 0.683 (0.814) | 1.46 | 3.1 Fold |
| | | 5:1 | 1.624 (0.225) | 0.62 | 7.4 Fold |
| | | 10:1 | 0.864 (0.107) | 1.16 | 3.9 Fold |
| | 3 | 1:1 | 2.415 (0.246) | 1.24 | 11.0 Fold |
| | | 5:1 | 2.977 (0.441) | 1.01 | 13.5 Fold |
| | | 10:1 | 2.431 (0.271) | 1.23 | 11.1 Fold |
| | 6 | 1:1 | 1.922 (0.416) | 3.12 | 8.7 Fold |
| | | 5:1 | 2.037 (0.401) | 2.95 | 9.3 Fold |
| | | 10:1 | 2.064 (0.227) | 2.91 | 9.4 Fold |
| Fmoc₁₀ | 1 | 1:1 | 0.198 (0.016) | 5.05 | 0.00 Fold |
| | | 5:1 | 1.638 (0.461) | 0.61 | 7.4 Fold |
| | | 10:1 | 1.737 (0.100) | 0.58 | 7.9 Fold |
| | 3 | 1:1 | 2.012 (0.598) | 1.49 | 9.1 Fold |
| | | 5:1 | 3.320 (0.085) | 0.90 | 15.1 Fold |
| | | 10:1 | 3.151 (0.318) | 0.95 | 14.3 Fold |
| | 6 | 1:1 | 3.609 (0.326) | 1.66 | 16.4 Fold |
| | | 5:1 | 4.463 (0.603) | 1.34 | 20.3 Fold |
| | | 10:1 | 4.792 (0.655) | 1.25 | 21.8 Fold |
| Dansyl₅ | 1 | 1:1 | 1.067 (0.371) | 0.94 | 4.85 Fold |
| | | 5:1 | 1.619 (0.201) | 0.62 | 7.4 Fold |
| | | 10:1 | 2.131 (0.212) | 0.47 | 9.7 Fold |
| | 3 | 1:1 | 0.896 (0.101) | 3.35 | 4.1 Fold |
| | | 5:1 | 1.779 (0.239) | 1.69 | 8.1 Fold |
| | | 10:1 | 4.867 (0.505) | 0.62 | 22.1 Fold |
| | 6 | 1:1 | 1.212 (0.265) | 4.95 | 5.5 Fold |
| | | 5:1 | 2.695 (0.777) | 2.23 | 12.3 Fold |
| | | 10:1 | 1.484 (0.263) | 4.04 | 6.7 Fold |
| Dansyl₁₀ | 1 | 1:1 | 2.262 (0.187) | 0.44 | 10.3 Fold |
| | | 5:1 | 2.534 (0.777) | 0.39 | 11.5 Fold |
| | | 10:1 | 2.727 (0.229) | 0.37 | 12.4 Fold |
| | 3 | 1:1 | 2.541 (0.386) | 1.18 | 11.6 Fold |
| | | 5:1 | 4.230 (0.291) | 0.71 | 19.2 Fold |
| | | 10:1 | 3.630 (0.604) | 0.83 | 16.5 Fold |
| | 6 | 1:1 | 2.732 (0.177) | 2.20 | 12.4 Fold |
| | | 5:1 | 4.325 (0.919) | 1.39 | 19.7 Fold |
| | | 10:1 | 31.815 (3.008) | 0.19 | 144.6 Fold |

FIG. 24 shows the maximum concentration of prednisolone solubilised by each polymer; Dansyl₁₀ was the most effective system as 31.82 mgmL⁻¹ of drug was solubilised which was 100-fold improvement of its aqueous solubility giving and excipient:drug ratio of only 0.19. The solubilisation capacity QCh₅ (2.05 mgmL⁻¹) was significantly lower than for Ch₅ (7.05 mgmL⁻¹) consistant with Propofol; therefore no further analysis was carried out on the quaternized compounds.

### Solubilisation of Griseofulvin

When comparing the solubilisation data for propofol and prednisolone, two polymers consistently stood out, these were Ch₅ and Dansyl₁₀. The concentration and drug:polymer ratio was 6 mgmL⁻¹ and 10:1 for both the comb-shaped polymers. All subsequent solubilisation studies used these two polymers at their optimal conditions (6 mgmL⁻¹, 10:1 drug:polymer).

The retention time for griseofulvin detection on the HPLC was around 9 mins (FIG. 25). Ch₅ and Dansyl₁₀ were capable of solubilising 1.2 and 16.7 mgmL⁻¹ griseofulvin (Table 6). This was an increase in the aqueous solubility (0.03 mgmL⁻¹) by 40-fold and 557-fold respectively. The Dansyl₁₀ had an extremely excipient:drug ratio of 0.34.

TABLE 6
Drug loading capacity of griseofulvin in modified polymers
determined using reverse phase HPLC for each drug:polymer
ratio n = 3, resulting excipient:drug ratio's and degree of
solubility increase from aqueous solubility of drug.

| | | | Concentration of | | Increase |
|---|---|---|---|---|---|
| | Polymer | | Griseofulvin | Excipient:Drug | from |
| | Conc. | Drug:Polymer | solubilised | Ratio after | aqueous |
| Polymer | mgmL⁻¹ | Ratio | mgmL⁻¹ ± SD | solubilisation | solubility |
| Ch₅ | 6 | 10:1 | 1.200 (0.017) | 5.00 | 40 Fold |
| Dansyl₁₀ | 6 | 10:1 | 16.710 (4.121) | 0.34 | 557 Fold |

### Solubilisation of Etoposide

Etoposide was detected at 229 nm at around 5 mins (FIG. 26). Table 7 shows that Ch₅ and Dansyl₁₀ were capable of solubilising up to 1.0 and 1.9 mgmL⁻¹ of etoposide respectively. Consistent with the previous solubilisations studies, the Dansyl₁₀ was the most efficient with a 13-fold increase from the aqueous solubility of the drug (0.15 mgmL⁻¹).

TABLE 7
Drug loading capacity of Etoposide in modified polymers
determined using reverse phase HPLC for each drug:polymer ratio n = 3,
resulting excipient:drug ratio's and degree of solubility increase from
aqueous solubility of drug.

| | | | Concentration of | | Increase |
|---|---|---|---|---|---|
| | Polymer | | Etoposide | Excipient:Drug | from |
| | Conc. | Drug:Polymer | solubilised | Ratio after | aqueous |
| Polymer | mgmL⁻¹ | Ratio | mgmL⁻¹ ± SD | solubilisation | solubility |
| Ch₅ | 6 | 10:1 | 1.020 (0.040) | 5.88 | 7 Fold |
| Dansyl₁₀ | 6 | 10:1 | 1.890 (0.011) | 3.17 | 13 Fold |

### Solubilisation of BNIPDaoct

FIG. 27 shows an HPLC spectra for the drug at a relatively low concentration (0.039 mgmL⁻¹ in DMSO:Water (50:50 v/v)). A signal peak is observed at around 7 mins.

Ch₅ was capable of solubilising 0.3 mgmL⁻¹ of the drug (1 mgmL⁻¹ polymer and 1:1 drug:polymer ratio) (Table 7). Previously all studies to find an aqueous solubility of BNIPDaoct yielded no results as the aqueous solubility was negligible. Therefore no degree of increase could be calculated. The excipient:drug ratio was 3.33. However on loading the Ch₅ at higher polymer concentrations and higher drug:polymer ratios the HPLC spectra changed dramatically. FIG. 28a to e shows the HPLC spectra of 1 mgmL⁻¹ Ch₅ at 1:1, 5:1 and 10:1 drug:polymer ratios.

TABLE 8
Drug loading capacity of BNIPDaoct in modified polymers
determined using reverse phase HPLC for each drug:polymer ratio n = 3,
resulting excipient:drug ratio's and degree of solubility increase from
aqueous solubility of drug.

| | | | Concentration of | | Increase |
|---|---|---|---|---|---|
| | Polymer | | BNIPDaoct | Excipient:Drug | from |
| | Conc. | Drug:Polymer | solubilised | Ratio after | aqueous |
| Polymer | mgmL⁻¹ | Ratio | mgmL⁻¹ ± SD | solubilisation | solubility |
| Ch₅-PAA | 1 | 1:1 | 0.3 | 3.33 | No intrinsic |
| | | | | | value |

As the drug:polymer ratio was increased the concentration of BNIPDaoct solubilised was also increased. As the concentration of solubilised drug increased (even upon extensive dilution) additional peaks were observed on the spectra making it inaccurate for determination of drug loading concentration. These extra peaks were witnessed when using the Dansyl₁₀ polymer also. Therefore no further solubilisation data for Ch₅ or Dansyl₁₀ could be deduced.

It appears that at higher drug concentration the presence of isomers is more probable, these isomers may have been retained at different times on the HPLC column, giving rise to extra peaks. However, since these peaks were not witnessed on the calibration, accumulative analysis would be inaccurate.

### Photon Correlation Spectroscopy

The hydrodynamic size for the optimal formulations of each drug was determined by photon correlation spectroscopy (Table 9).

TABLE 9
Photon correlation spectrometery size analysis of optimal Ch₅ and
Dansyl₁₀ formulations ± SD, n = 3.

| | | | Drug:Polymer | Size nm | PDI |
|---|---|---|---|---|---|
| Polymer | | Drug | Ratio | mean ± SD | mean ± SD |
| Ch₅ | 6 mgmL⁻¹ | — | — | 296 (3.637) | 0.285 (0.004) |
| Ch₅ | 6 mgmL⁻¹ | Propofol | 10:1 | 666 (79.481) | 0.376 (0.025) |
| Ch₅ | 6 mgmL⁻¹ | Prednisolone | 10:1 | 304 (3.533) | 0.318 (0.014) |
| Ch₅ | 6 mgmL⁻¹ | Griseofulvin | 10:1 | 284 (5.542) | 0.209 (0.012) |
| Ch₅ | 6 mgmL⁻¹ | Etoposide | 10:1 | | |
| Ch₅ | 1 mgmL⁻¹ | BNIPDaoct | 1:1 | | |
| Dansyl₁₀ | 6 mgmL⁻¹ | — | — | 126 (8.382) | 0.465 (0.022) |
| Dansyl₁₀ | 6 mgmL⁻¹ | Propofol | 10:1 | 608 (49.37) | 0.385 (0.009) |
| Dansyl₁₀ | 6 mgmL⁻¹ | Prednisolone | 10:1 | 350 (4.115) | 0.369 (0.007) |
| Dansyl₁₀ | 6 mgmL⁻¹ | Griseofulvin | 10:1 | 305 (6.874) | 0.254 (0.007) |
| Dansyl₁₀ | 6 mgmL⁻¹ | Etoposide | 10:1 | 886 (115.3) | 0.425 |

The Ch₅-PAA alone had a size of 295 nm. The pattern of the size measurements followed the pattern of the drug loading capability. The propofol loaded self assembly had the largest solubilisation capacity (7.80 mgmL⁻¹) and size (666 nm), followed by the prednisolone with solubilisation capacity of 7.05 mgmL⁻¹ and a size of 304 nm. The Griseofulvin self assemblies were only capable of solubilising 1.2 mgmL⁻¹ with a size of only 284 nm.

The size Dansyl₁₀-PAA was 126 nm; this was significantly smaller than the drug loaded self assemblies. The inner hydrophobic core possessed the ability to expand to a high degree and therefore encapsulate a high drug concentration. The propofol loaded system had the greatest size of 608 nm, followed by the prednisolone loaded system 350 nm, with Griseofulvin having the smallest size of 305 nm.

### Transmission Electron Microscopy

Optimal formulations were sent for TEM imaging. The TEM micrographs of the Ch₅ and Ch₅ formulations (FIG. 29a to e) show clearly the nanoparticles formed. The Ch₅ alone appears to form solid nanoparticles at 6 mgmL⁻¹, however once loaded with propofol a micellar structure is present. The Prednisolone and Griseofulvin loaded self assemblies appear to be colloidal dispersions.

The TEM micrographs of Dansyl₁₀ and Dansyl₁₀ formulations show that Nanoparticles are formed at 6 mgml⁻¹ of the polymer in solution, however on addition of propofol and griseofulvin, polymeric micelles appear to have formed. The formation of prednisolone loaded Dansyl₁₀ appear to be nano-particles also.

The size of particles on the micrographs confirms the results achieved by the photon correlation spectroscopy.

### FTIR of Freeze Dried Formulations

The freeze dried preparations were run on the FTIR to enable visualisation of the functional groups present and to confirm the presence of the drug and polymer.

The characteristic band at 3365 cm⁻¹ on the Ch₅ spectra (FIG. 31) showed the presence of the secondary amine on the polymer backbone, the peak observed at 2923 cm⁻¹ was due to the C—H bond bending in the alkyl chain of the polymer backbone. The presence of the carbonyl peak at 1575 and 815 cm⁻¹ were due to the attachment of the cholesterol moiety onto the PAA backbone. All the peaks were assigned in Table 10.

TABLE 10
Peak bandwidth assignment occurring on FTIR spectra of Ch₅
using diamond powder tip (20scans).

| Polymer | Bandwidth cm⁻¹ | Bond type | Functional Group |
|---|---|---|---|
| Ch₅ | 3365 | N—H | 2° Amine |
| | 1575 | | |
| | 2923 | C—H Bend | Alkyl |
| | 1460 | | |
| | 1575 | C═O stretch | Carbonyl |
| | 1311 | C—C Bend and Stretch | Alkyl |
| | 815 | C—O Stretch | |

FIG. 32 shows the FTIR spectra obtained from the Ch₅ and propofol formulation. The presence of the O—H peak at 3600 cm⁻¹ and the ortho substituted aromatic benzene peaks at 2100 and 748 cm⁻¹ were resultant from the functional groups present on the propofol moiety. This indicated that propofol had been successfully encapsulated in the formulation. All peak assignments can be seen in Table 11.

TABLE 11
Peak bandwidth assignment occurring on FTIR spectra of Ch₅ +
propofol formulation using diamond powder tip (20scans).

| | Bandwidth | | |
|---|---|---|---|
| Formulation | cm⁻¹ | Bond type | Functional Group |
| Ch₅ + | 3600 | O—H | Hydroxyl |
| propofol | 3300 | N—H | 2° Amine |
| | 1640-1560 | | |
| | 3100 | C—H | Aromatic |
| | 748 | | Ortho subs benzene |
| | 2962 | C—H Bend | Alkyl |
| | 1460 | | |
| | 1600 | C═O stretch | Carbonyl |
| | 1311 | C—C Bend and Stretch | Alkyl |
| | 815 | C—O Stretch | |

Peak assignment of the FTIR spectra obtained for the Ch₅ and prednisolone formulation (FIG. 33) confirmed the presence of the drug in formulation. This was due to the presence of the hydroxyl band at O—H (3404 cm⁻¹) and the C═O (1720 cm⁻¹) of the six membered carbonyl ring, these groups were not present in the spectra for Ch₅ alone and therefore can be attributed to the drug molecule. Full peak assignment can be viewed in Table 12.

TABLE 12
Peak bandwidth assignment occurring on FTIR spectra of Ch₅ +
prednisolone formulation using diamond powder tip (20scans).

| | Bandwidth | | |
|---|---|---|---|
| Formulation | cm⁻¹ | Bond type | Functional Group |
| Ch₅ + | 3404 | O—H | Hydroxyl |
| prednisolone | 1656 | N—H | 2° Amine |
| | 900-850 | C—H | Aromatic |
| | | | Subs benzene |
| | 2923 | C—H Bend | Alkyl |
| | 1460 | | |
| | 1720 | C═O | Carbonyl 6 membered |
| | | | ring |
| | 1311 | C—C Bend and | Alkyl |
| | | Stretch | |
| | 950 | C—O Stretch | |

FIG. 34 gives confirmation of the successful encapsulation of griseofulvin within the self assemblies. This can be deduced due to extra peaks being present at 3359 cm⁻¹ (O—H), 1750 cm⁻¹ (C═O), 800 cm⁻¹ (Aromatic substituted benzene) and a weak peak at 550 cm⁻¹. These extra peaks correlate well with the functional groups present on the griseofulvin moiety. For all peak assignments see Table 13.

TABLE 13
Peak bandwidth assignment occurring on FTIR spectra of Ch₅ +
griseofulvin formulation using diamond powder tip (20scans).

| | Bandwidth | | |
|---|---|---|---|
| Formulation | cm⁻¹ | Bond type | Functional Group |
| Ch₅ + | 3359 | O—H | Hydroxyl |
| griseofulvin | 1620 | N—H | 2° Amine |
| | 800 | C—H | Aromatic |
| | | | Subs benzene |
| | 2941 | C—H Bend | Alkyl |
| | 1460 | | |
| | 1750 | C═O | Carbonyl 5 membered |
| | | | ring |
| | 1600 | C═O stretch | Carbonyl |
| | 1500 | C—C Bend and | Alkyl |
| | | Stretch | |
| | 950 | C—O Stretch | |
| | 550 | C—Cl | Chloro alkane |

The characteristic band at 3373 cm⁻¹ on the Dansyl₁₀ spectra (FIG. 35) showed the presence of the secondary amine on the polymer backbone, the peak observed at 3070 cm⁻¹ was due to the C—H bond of the aromatic substituted benzene of the dansyl moeity. The presence of the C═C stretch at 1450 cm⁻¹ (aromatic)) and the C—N stretch (1070 cm⁻¹) confirmed the attachment of the dansyl moiety onto the PAA backbone. All the peaks were assigned in Table 14.

TABLE 14
Peak bandwidth assignment occurring on FTIR spectra of
Dansyl₁₀ using diamond powder tip (20scans).

| | Bandwidth | | |
|---|---|---|---|
| Polymer | cm⁻¹ | Bond type | Functional Group |
| Dansyl₁₀ | 3373 | N—H | 2° Amine |
| | 3070 | C—H | Aromatic |
| | 626, 792 | | Subs. benzene |
| | 2920 | C—H Bend | Alkyl |
| | 1460 | | |
| | 1500 | C—C Bend and Stretch | Alkyl |
| | 1450 | C═C Stretch | Aromatic |
| | 1070 | C—N Stretch | Aliphatic amine |

FIG. 36 shows the FTIR spectra obtained from the Dansyl₁₀ and propofol formulation. The presence of the O—H peak at 3419 cm⁻¹ and the ortho substituted aromatic benzene peaks at 750 cm⁻¹ were resultant from the functional groups present on the propofol moiety. This indicated that propofol had been successfully encapsulated in the formulation. All peak assignments can be seen in Table 15.

TABLE 15
Peak bandwidth assignment occurring on FTIR spectra
of Dansyl₁₀ and propofol formulation using
diamond powder tip (20scans).

| Polymer | Bandwidth cm⁻¹ | Bond type | Functional Group |
|---|---|---|---|
| Dansyl₁₀ + | 3419 | O—H | Hydroxyl |
| propofol | 1640-1560 | N—H | 1° Amine |
| | 750 | C—H | Aromatic |
| | | | Ortho Subs. benzene |
| | 2960 | C—H Bend | Alkyl |
| | 1450 | C═C Stretch | Aromatic |
| | 1095 | C—N Stretch | Aliphatic amine |
| | 950 | C—O Stretch | |

Peak assignment of the FTIR spectra obtained for the Dansyl₁₀ and prednisolone formulation (FIG. 37) confirmed the presence of the drug in formulation. This was due to the presence of the hydroxyl band at O—H (3413 cm⁻¹) and the C═O (1720 cm⁻¹) of the six membered carbonyl ring, these groups were not present in the spectra for Dansyl₁₀ alone and therefore can be attributed to the drug molecule. Full peak assignment can be viewed in Table 12.

TABLE 16
Peak bandwidth assignment occurring on FTIR spectra of
Dansyl₁₀ and prednisolone formulation using
diamond powder tip (20scans).

| | Bandwidth | | |
|---|---|---|---|
| Polymer | cm⁻¹ | Bond type | Functional Group |
| Dansyl₁₀ + | 3413 | O—H | Hydroxyl |
| prednisolone | 1656 | N—H | 1° Amine |
| | 900-850 | C—H | Aromatic |
| | | | Subs. benzene |
| | 2927 | C—H Bend | Alkyl |
| | 1720 | C═O Stretch | Carbonyl 6 - membered |
| | | | ring |
| | 1450 | C═C Stretch | Aromatic |
| | 950 | C—O Stretch | |

FIG. 38 gives confirmation of the successful encapsulation of griseofulvin within the self assemblies. This can be deduced due to extra peaks being present at 3377 cm⁻¹ (O—H), 1750 cm⁻¹ (C═O of Aromatic substituted benzene) and a weak peak at 750 cm⁻¹. These extra peaks correlate well with the functional groups present on the griseofulvin moiety. For all peak assignments see Table 17.

TABLE 17
Peak bandwidth assignment occurring on FTIR spectra of
Dansyl₁₀ and griseofulvin formulation using diamond
powder tip (20scans).

| | Bandwidth | | |
|---|---|---|---|
| Polymer | cm⁻¹ | Bond type | Functional Group |
| Dansyl₁₀ + | 3377 | O—H | Hydroxyl |
| griseofulvin | 1587 | N—H | 1° Amine |
| | 900-850 | C—H | Aromatic |
| | | | Subs. benzene |
| | 2921 | C—H Bend | Alkyl |
| | 1585 | | |
| | 1750 | C═O Stretch | Carbonyl 6 - |
| | | | membered ring |
| | 1500 | C—C Bend and Stretch | Alkyl |
| | 1450 | C═C Stretch | Aromatic |
| | 1150 | C—N Stretch | Aliphatic amine |
| | 750 | C—Cl | Choroalkane |

### In Vitro Drug Release

The release of the drugs from the optimal formulations was carried out in sink condition over 96 hrs at 37° C. in Phosphate Buffered Saline (PBS).

FIG. 39 shows drug release from the Ch₅ formulations. All the Ch₅ formulations were able to achieve sustained drug release over period 48-72 hrs. Within the first 7 hrs, 5% of the propofol had been released whilst 55% of the prednisolone and only 20% of the griseofulvin had been released. The propofol had reached 100% release after 48 hrs, after 72 hrs the prednisolone and griseofulvin were 100% released.

FIG. 40 shows the release profiles of the drugs from the Dansyl₁₀ formulations. All the formulations achieved sustained release up to 96 hrs. After 72 hrs the propofol and griseofulvin had been completely released from formulation, however the prednisolone was retained in formulation until 96 hrs when it was fully released.

### Stability of Formulations

The formulations were stored in a dark airtight container for 4 wks. Over this time the formulations were analysed for total drug content and hydrodynamic size was determined using photon correlation spectroscopy. The formulations were initially stored in two forms; as liquid formulations and as freeze dried pellets (reconstituted with water and sonicated). However, on initial testing of the freeze dried pellets, it was deduced that all the propofol of the formulations had been lost in the freeze drying process, perhaps due to the volatile nature of this drug.

On commencement of testing it was apparent that the prednisolone and griseofulvin liquid preparations were forming viscous gels over time. These gels were too thick to be analysed.

The stability data shown (FIG. 41-41, Table 18-19) are therefore of liquid propofol preparations and freeze dried prednisolone and griseofulvin preparations.

Over the 4 wk period propofol appeared to be gradually lost from the Ch₅ formulation (FIG. 41) from 0-30% (0-4 wk). The size data for the propofol formulation correlates well with the drug loss. The size of the self assemblies are reduced from 666-239 nm as the drug content decreases (0-4 wk) (Table 18). The initial loss of prednisolone and griseofulvin (15% and 10% respectively) experienced from the Ch₅ formulation was through the freeze drying process. However no further notable loss was apparent over the 4 wk period. The size of the self assemblies did not considerably change however the poly-dispersity index for both formulations was relatively high, this indicated the aggregates were not of uniform size.

TABLE 18
Size data for Ch₅ formulations over a 4 wk period ± SD, n = 3.
Propofol samples stored as solution, Prednisolone samples stored a
freeze dried pellets and reconstituted with water. All samples stored in
air-tight container at room temperature in darkened conditions

| | | Photon correlation spectroscopy | |
|---|---|---|---|
| | | Size nm | PDI |
| Formulation | Time wks | mean ± SD | mean ± SD |
| 6 mgmL⁻¹ Ch₅ | 0 | 666 (79.418) | 0.376 (0.025) |
| 1:10 Propofol | 1 | 394 (49.048) | 0.340 (0.066) |
| | 2 | 367 (79.811) | 0.282 (0.104) |
| | 3 | 264 (5.356) | 0.285 (0.025) |
| | 4 | 239 (3.804) | 0.237 (0.023) |
| 6 mgmL⁻¹ Ch₅ 1:10 | 0 | 907 (37.470) | 0.560 (0.044) |
| Prednisolone | 1 | 881 (52.818) | 0.477 (0.069) |
| | 2 | 1006 (14.962) | 0.584 (0.093) |
| | 3 | 809 (18.540) | 0.375 (0.009) |
| | 4 | 883 (37.906) | 0.399 (0.038) |
| 6 mgmL⁻¹ Ch₅ 1:10 | 0 | 658 (20.932) | 0.694 (0.027) |
| Griseofulvin | 1 | 437 (34.453) | 0.674 (0.156) |
| | 2 | 536 (24.278) | 0.593 (0.089) |
| | 3 | 464 (10.313) | 0.433 (0.035) |
| | 4 | 424 (5.352) | 0.382 (0.006) |

The stability profiles from the Dansyl₁₀ formulations in FIG. 42 show similar patterns to the release from the Ch₅ formulations. The propofol had a cumulative loss over the 4 wk period, however only 10% was released (in comparison with Ch₅ and propofol 30%). The size of the aggregates in the formulations remained consistent across the 4 wk period (Table 19). The initial loss of prednisolone and griseofulvin (10% and 20% respectively) from the freeze drying process was also observed which was consistent with the Ch₅ formulations. However the griseofulvin content did decrease over the 4 wk period with 40% being lost after week four. Both the prednisolone and griseofulvin self assemblies increased in size across the 4 wk period from 462-829 nm and from 545-1055 nm respectively. The polydispersity index's also varied greatly.

TABLE 19
Size data for Dansyl₁₀ formulations over a 4 wk period ± SD, n = 3.
Propofol samples stored as solution, Prednisolone samples stored a
freeze dried pellets and reconstituted with water. All samples stored in
air-tight container at room temperature in darkened conditions

| | | Photon correlation spectroscopy | |
|---|---|---|---|
| | | Size nm | PDI |
| Formulation | Time wks | mean ± SD | mean ± SD |
| 6 mgmL⁻¹ | 0 | 608 (49.37) | 0.385 (0.009) |
| Dansyl₁₀ 1:10 | 1 | 631 (140.922) | 0.300 (0.083) |
| Propofol | 2 | 585 (90.113) | 0.349 (0.059) |
| | 3 | 741 (133.671) | 0.386 (0.009) |
| | 4 | 677 (124.107) | 0.324 (0.063) |
| 6 mgmL⁻¹ Dansyl₁₀ | 0 | 462 (29.441) | 0.422 (0.111) |
| 1:10 Prednisolone | 1 | 703 (4.561) | 0.499 (0.235) |
| | 2 | 715 (43.736) | 0.646 (0.254) |
| | 3 | 618 (87.825) | 0.552 (0.102) |
| | 4 | 829 (134.476) | 0.970 (0.541) |
| 6 mgmL⁻¹ Dansyl₁₀ | 0 | 545 (18.785) | 0.586 (0.022) |
| 1:10 Griseofulvin | 1 | 687 (84.742) | 0.131 (0.105) |
| | 2 | 806 (20.731) | 0.247 (0.087) |
| | 3 | 1090 (83.781) | 0.541 (0.101) |
| | 4 | 1055 (75.434) | 0.513 (0.029) |

The driving force behind the encapsulation of hydrophobic drugs inside the core of polymeric micelles is a basic energetic principle. It is energetically favourable for hydrophobic drugs to shield themselves from aqueous environments. As the polymeric micelle spontaneously assemblies into its core-shell structure, the smaller lipophilic drug molecules accumulate within the hydrophobic region where they remain physically entrapped as the micelle spontaneously breaks and forms in dynamic equilibrium. Model hydrophobic drugs were encapsulated inside the modified PAA polymers and analysed for their drug content. The modified polymers all possessed the ability to encapsulate practically insoluble drugs within their hydrophobic core. The results confirm that varying the hydrophobic group directly impacts the self assembly loading capacity. This is perhaps due to the chemical structure of the group. The small planar structure of Fmoc, enabled excimer formation to occur, where stacking of the molecules occurred, this was also witnessed in the Naphthalene modified polymer. The close stacking of these molecules may limit the number of drug molecules entering the hydrophobic core, thus making them less effective; however, this may differ with differing drug architecture. The more bulky or rigid hydrophobic molecules such as Cholesterol or Dansyl may experience some steric hindrance when in close proximity to each other, however small drug molecules may be able to ‘slot’ into such spaces and maximise their loading concentration.

Hydrophilic modification of the polymer backbone appeared to decrease the maximum concentration of propofol solubilised. However, visual examination of the formulations before filtration would suggest otherwise. Clear solutions were formed upon sonication which would suggest drug was totally solubilised. Perhaps this could be due to the positive charge forming weaker or larger self assemblies encapsulating the propofol which were removed during the filtration process.

Ch₅ and Dansyl₁₀ proved to be the best solubilising agents across the library of polymers synthesised. The solubilising potential of these amphiphilic polymers was consistent across the range of model drugs used. This indicates that the polymers are non-drug selective and therefore capable of solubilising any hydrophobic drug and are not specific to one molecular type. This is an excellent property to possess in a drug delivery system as it is generic and can be used across a broad range of applications.

Overall it was observed for the Ch₅ and Dansyl₁₀ formulations, that higher polymer concentration achieved greatest solubility of the hydrophobic drugs, this was due to the fact that more micelles would have been present in solution, therefore a higher degree of solubilisation could be achieved. The higher the drug:polymer ratio also achieved increased solubility, this could be due to higher hydrophobicity being present at higher concentrations, therefore the driving force for solubilisation in the hydrophobic core of the micelles was greater resulting in higher solubilisation concentration of the drug. The excipient:drug ratios were calculated. Solubilisation was achieved at excipient:drug ratios as small as 0.13 (6 mgmL⁻¹ Dansyl₁₀, 10:1 propofol:polymer ratio). It is desirable to use low excipient:drug ratios when solubilising a drug, the system is not only more efficient and cost effective, but it also minimises any harmful or uncomfortable side effects to the patient.

The ability of one polymer to load different drugs with differing efficiency was observed. This was down to the architecture of the drugs being loaded. Planar drugs have the ability to stack closely together, allowing higher concentrations to become encapsulated within the hydrophobic core. Bulkier or more rigid molecules find it more difficult to accumulate together in high concentration as steric hindrance prevents this occurring, meaning the encapsulation efficiency is decreased.

In the case of propofol, the small structural size (FIG. 15), allows the molecules to fit closely together without much steric hindrance thus maximising the concentrations being encapsulated (FIG. 21).

The polymeric micelles also encapsulated prednisolone, to high degree (FIG. 24). The chemical structure of prednisolone (FIG. 16), although the most bulky, is still mostly flexible, thus allowing the structure to bend appropriately to accommodate the close proximity of other molecules of the drug. This property increases the encapsulation efficiency. Prednisolone was solubilised by Dansyl₁₀ (31.82 mgmL⁻¹) achieving a 100 fold improvement of its aqueous solubility.

The griseofulvin (FIG. 17) solubilised was 400-fold better than the aqueous solubility, however when compared to the propofol and the prednisolone, a much lower concentration of drug was encapsulated (Table 6). Although the structure of griseofulvin is relatively small, the amount of double bonds present makes the molecule almost inflexible. The rigidity of this molecule means it cannot change to accommodate other molecules in close proximity and thus steric prevents higher loading occurring.

Etoposide is a relatively large drug molecule, it is made up of four conjugated five and six membered rings with other ring systems branched off. The branching allows for a degree of flexibility within the structure. Ch₅ (1.02 mgmL⁻¹) and Dansyl₁₀ (1.89 mgmL⁻¹) were capable of increasing the aqueous solubility of etoposide by 7-fold and 13-fold respectively. However this could be accounted for by the larger molecular size. It would be expected that a lower drug loading concentration could be achieved as the polymeric aggregate is not capable of infinite expansion.

The drug loading of BNIPDaoct could not be accurately carried out due to potential isomers being present at higher concentration causing peak variances on the HPLC spectra. However at 1 mgmL⁻¹ Ch₅ drug was solubilised. BNIPDaoct is an anticancer drug, when intrinsic aqueous solubility was carried out for this drug no value was achieved. The aqueous solubility was negligible, therefore the percentage increase from the aqueous value cannot be calculated as the increase in infinite.

The varied sizes of the drug loaded polymers reinforced the theory that the molecular architecture of the hydrophobic drug hugely affects its ability to load within the micelle core. In Vitro release profiles showed that all the Ch₅ and Dansyl₁₀ formulations achieved sustained release between 48-72 hrs. The release profiles did not show any correlation with the drug loading profile.

The stability of a formulation is most important in order for it to be applicable to the commercial world. The formulations all went through stability testing. The results reinforce the fact that different formulations using the same excipient do not all require the same storage conditions. The stability of each formulation differed, the propofol formulations stored as liquids appeared to slowly lose drug content over the 4 wk period up to a maximum of 30% (Ch₅) and 10% (Dansyl₁₀). The prednisolone and griseofulvin formulations on the whole appeared to be stable across the 4 wk period. The major drug loss for these formulations occurred in the initial freeze drying process, however with careful optimisation this drug loss could perhaps be minimised. Interestingly the sizes of the self assemblies were significantly larger after freeze drying and reconstitution, this phenomenon may arise as a result of damage to the polymer during the freeze-drying process or due to the nano-aggregates accumulating in a different formation. Ch₅ and Dansyl₁₀ have shown excellent drug loading capacities and release profiles.

### In Vivo Oral Absorption of Griseofulvin

All solutions were made immediately prior to in vivo administration.

### Preparation of Griseofulvin in Water

Griseofulvin (2 mgmL⁻¹) was added to doubly distilled water and sonicated for 10 min to ensure maximum solubilisation of the drug had occurred. The solution was filtered using 0.45 μm syringe filters with prefilters to remove any excess undissolved drug.

### Preparation of Ch₅ and Dansyl₁₀ Griseofulvin Formulations

Ch₅ polymer solution (6 mgmL⁻¹) was prepared by dissolving the polymer in water followed by probe sonication (10 min). Griseofulvin (60 mgmL⁻¹) was added to the Ch₅ solution and sonicated for a further 10 min to ensure maximum drug solubilisation had occurred. The solution was filtered using 0.45 μm syringe filters with prefilters to remove any excess undissolved drug. The amount of solubilised griseofulvin was 1.2 mgmL⁻¹. The Dansyl₁₀, griseofulvin formulation was prepared as described above (16.71 mgmL⁻¹) except a 1 in 5 dilution was then performed of the polymer-drug formulation, to adjust the dosage and enable direct comparison of the results with the Ch₅, griseofulvin formulation.

### In Vivo Oral Administration and Evaluation of Griseofulvin Absorption

18 male sprague dawley rats (280 g) were separated into four groups (n=4 groups or n=3, ave controls) and their food was withdrawn over night (18 h), the rats had free access to water at all times. The rats were orally dosed with a griseofulvin suspension in water and two griseofulvin formulations (11.8 mgKg⁻¹) via oral gavage. The formulations were prepared as previously described. The rats were monitored to evaluate their behaviour immediately after administration and throughout the investigation. Blood samples (approximately 100 μL) were collected using 300 μL microvettes (Microvette®CB300, Vet Tech Solutions, UK) at various time points (1, 4 and 7 h) via tail vein venesection. After the first time point (1 h) food was given to the rats. After 24 h the rats were sacrificed using a carbon dioxide chamber and an immediate heart puncture was carried out to retrieve the blood. All blood samples were centrifuged at 2000 rpm for 10 min and the plasma was pipetted off into separate eppendorf tubes. The plasma samples were frozen until ready for analysis. Polymers alone were used as the controls.

Plasma (100 μL) was diluted with 250 μL acetonitrile and vortexed for 30 sec. The mixture was then centrifuged at 3000 rpm for 10 min. The supernatant was collected and 50 μL was injected onto the column. The griseofulvin content of the plasma was determined by HPLC analysis. A RP Zorbax ODS 250 mm×46 mm×5 μm HPLC column (Hichrom, UK) was used with a flow rate of 2 mLmin⁻¹ (50:50 v/v acetonitrile:water) in an isocratic mode (Varian LC, Varian UK). The resultant peak at 3 min was analysed at 260 nm (excitation) and 389 nm (emission) using a fluorescent detector (Varian LC, Varian UK). The amount of griseofulvin present in the samples was determined by comparing to a standard calibration carried out previously with griseofulvin spiked blank plasma samples (1.9 μgmL⁻¹-10 μgmL⁻¹), R2=0.992. The statistical significance of the results was assessed using two-way analysis on variance ANOVA and Dunnett multiple comparison t-test via SPSS 13.0 for Windows.

### In Vivo Oral Absorption of Griseofulvin

Griseofulvin in water and two griseofulvin formulations (using Ch₅ and Dansyl₁₀) were administered to male Sprague dawley rats via oral gavage. The rats were constantly monitored visually to ensure that no gross toxicity was experienced, causing pain or discomfort to the animals. The rats were fully alert and appeared comfortable after the dosing with the exception to one. The level of griseofulvin present in plasma samples of the rats was determined at 1, 4, 7 and 24 h time points by HPLC analysis (FIG. 44). At all time points the griseofulvin formulation appeared to have higher plasma drug levels than the griseofulvin in water. The griseofulvin concentration at each time point was found to be significantly higher than the griseofulvin in water control (p<0.001). The CMax (concentration at which the maximum level of drug was absorbed) of the griseofulvin in water was at 1 h, after this time no more drug was absorbed. The Ch₅, griseofulvin showed the greatest absorption, with as much as 17.059 μgmL⁻¹ found in the plasma. The tMax (time at which the maximum concentration was observed) occurred at 4 h for this formulation. This indicated that the formulation was stable within the stomach enabling absorption possibly in the small intestine and into the blood plasma. In contrast, the tMax of Dansyl₁₀, griseofulvin formulation was after 1 h with a CMax of 9.752 μgmL⁻¹. The lower absorption of the Dansyl₁₀ formulation could be due to the dilution carried out in the preparation stages resulting in lower polymer concentration leading to instability within the stomach, whereby, the formulation prematurely released its payload hindering efficient absorption across the GI tract over sustained periods of time. This observation correlates well with the larger critical association concentration (CAC) of Dansyl₁₀ (0.25 mgmL⁻¹) compared to Ch5 (0.093 mgmL⁻¹).

Various modifications and variations to the described embodiments of the invention will be apparent to those skilled in the art without departing from the scope of the invention as described herein.

Although the invention has been described in connection with specific preferred embodiments, it should be understood that the invention as described should not be unduly limited to such specific embodiments.

1. A polymer having a structure according to the following formula: 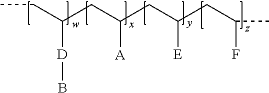
wherein:
A represents a hydrophilic group;
B represents a hydrophobic group;
D and E independently represent amine groups;
F represents an amine group, the amine group being substituted with a B group and an A group, or the amine group being a quaternary ammonium moiety; wherein W, X, Y and Z each independently have values greater than or equal to 1;
and wherein the hydrophobic group B is an aromatic group.

2. A polymer according to claim 1 wherein the values of W, X, Y and Z are each independently between 1 and 25.

3. A polymer according to claim 1 wherein the values of W, X, Y and Z are each independently between 1 and 10.

4. A polymer according to claim 1, wherein the polymer is based upon a polyallylamine (PAA) polymer.

5. A polymer according to claim 1, wherein the polymer is amphiphilic.

6. A polymer according to claim 1, wherein the polymer contains a chromophore.

7. A polymer according to claim 6 wherein the aromatic group of the hydrophobic group B comprises the chromophore.

8. A polymer according to claim 1, wherein the polymer is fluorescent.

9. A polymer according to claim 1, wherein the aromatic group of the hydrophobic group B is fluorescent.

10. A polymer having a structure according to the following formula: 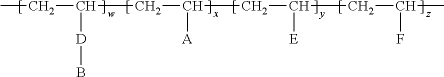
wherein:
D represents CH₂—NH;
B represents a hydrophobic, aromatic
A represents a CH₂-hydrophilic group;
E represents CH₂—NH₂;
F represents: 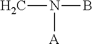
or a quaternary ammonium moiety wherein one of the substituents of the quaternary ammonium moiety is a B group.

11. A polymer according to claim 10 wherein the polymer is based upon a polyallylamine (PAA) polymer.

12. A polymer according to claim 11 wherein the polymer has an average molecular weight of between 10 and 70 kD.

13. A polymer according to claim 10, wherein the hydrophobic, aromatic group B is one or more of a bi-cyclic ring system, a tri-cyclic ring system, a phenyl group or alkylbenzene group.

14. A polymer according to claim 13 wherein the one or more bi-cyclic ring system, tri-cyclic ring system, phenyl group and alkylbenzene group is substituted.

15. A polymer according to claim 10, wherein the hydrophilic group represents a quaternary ammonium moiety with the structure:

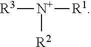

16. A polymer according to claim 15 wherein R¹, R² and R³ independently represent a hydrogen, alkyl, alkenyl, alkynyl, aryl, acyl, hydroxy alkyl, hydroxy acyl, polyethylene glycol or sugar group.

17. A polymer according to claim 1, wherein the hydrophobic aromatic group B is 5-dimethylamino-1-naphthalenesulfonyl (Dansyl), where Dansyl has the structure:

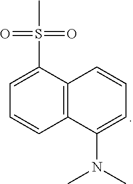

18. A polymer according to claim 1, wherein the hydrophobic aromatic group B is 9-fluorenylmethoxy carbonyl (Fmoc), where Fmoc has the structure:

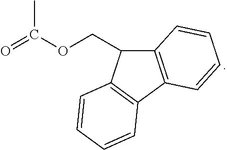

19. A polymer according to claim 1, wherein the hydrophobic aromatic group B is naphthalene (Naphth), where Naphth has the structure:

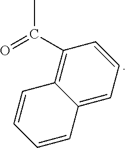

20. A polymer having the following structure: 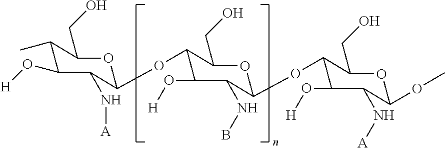
wherein A represents a CH₂-hydrophilic group; and B represents a hydrophobic, aromatic group.

21. A pharmaceutical composition comprising an active ingredient and a carrier, wherein the carrier is a polymer according to claim 1.

22. A pharmaceutical composition according to claim 21 further comprising an aqueous pharmaceutically acceptable vehicle.

23. A pharmaceutical composition according to claim 22, wherein the ratio of polymer to pharmaceutically acceptable vehicle is between 0.01 and 1:100 by weight/volume (w/v) (g/ml).

24. A pharmaceutical composition according to claim 22, wherein the pharmaceutically acceptable vehicle is distilled water.

25. A pharmaceutical composition according to claim 22, wherein the active ingredient has an aqueous solubility of between 0.001 and 0.2 mg/ml at a temperature of between 15 and 25° C.

26. A pharmaceutical composition according to claim 22, wherein the active ingredient is one or more of a drug, peptide, protein or polymer.

27. A method of providing a hydrophobic active ingredient to a subject, comprising administering to the subject a pharmaceutical composition comprising said hydrophobic active ingredient and a carrier, wherein the carrier comprises the polymer according to claim 1.

28. A method of providing a nucleic acid to a subject, comprising administering to the subject a pharmaceutical composition comprising said nucleic acid and a carrier, wherein the carrier comprises the polymer according to claim 1.

29. A method of preparing a pharmaceutical composition, comprising mixing a hydrophobic active ingredient with a polymer according to claim 1, whereby the solubility of the hydrophobic active ingredient in an aqueous media of the resulting pharmaceutical composition is increased relative to the solubility of the hydrophobic active ingredient alone.

30. A method of preparing a pharmaceutical composition, comprising mixing a nucleic acid with a polymer according to claim 1, whereby the solubility of the nucleic acid in a non-aqueous media of the resulting pharmaceutical composition is increased relative to the solubility of the nucleic acid alone.
